(19) United States
(12) Patent Application Publication
(10) Pub. No.: US 2026/0076628 A1
(43) Pub. Date: Mar. 19, 2026

(54) GLUCOSE MONITORING DEVICE

(71) Applicant: Custom TypeOne LLC, Stow, OH (US)

(72) Inventors: Chris Fawcett, Charlotte, NC (US); Jonathan Eric Fawcett, Stow, OH (US)

(21) Appl. No.: 19/400,151

(22) Filed: Nov. 25, 2025

Related U.S. Application Data
(63) Continuation-in-part of application No. 18/943,187, filed on Nov. 11, 2024.
(60) Provisional application No. 63/602,789, filed on Nov. 27, 2023.
(60) Provisional application No. 63/797,551, filed on Apr. 30, 2025.
(60) Provisional application No. 63/808,783, filed on May 20, 2025.

(51) Int. Cl. A61B 5/00; A61B 5/145

(52) U.S. Cl. CPC A61B 5/746; A61B 5/14532; A61B 5/742

(57) ABSTRACT

A system is provided comprising external device(s), tracking device(s) configured to generate blood glucose level data for tracked individual(s), and an alert device for management of alerts generated at the external device(s). The alert device comprises processor(s) and memory device(s). The memory device(s) comprise computer readable code that, when executed by the processor(s), causes the processor(s) to receive input data comprising blood glucose level data received from the tracking device(s), to determine an alert setting for an alert to be generated at an external device of the external device(s) based on the input data, and to generate a signal that causes the alert to be generated at the external device. The alert setting includes an identification of the external device where the alert is to be generated, an alert magnitude, and/or the alert type, with the alert type including an audible alert, a haptic alert, and/or a visual alert.

### CROSS-REFERENCE TO RELATED APPLICATIONS

This application claims priority to and is a continuation in part of U.S. patent application Ser. No. 18/943,187, entitled “Glucose Monitoring Alarm Device and Related Functionality,” which was filed Nov. 11, 2024, and which claims priority to U.S. Provisional Patent Application No. 63/602,789, which was filed Nov. 27, 2023, and which was entitled “Glucose Monitoring Alarm Device and Related Functionality.” This application also claims priority to U.S. Provisional Patent Application No. 63/797,551, filed Apr. 30, 2025, and entitled “Glucose Monitoring Device” and to U.S. Provisional Patent Application No. 63/808,783, filed May 20, 2025, and entitled “Glucose Monitoring Device.” Each of these applications are hereby incorporated by reference in their entireties for all purposes.

### FIELD

Embodiments relate generally to glucose monitoring systems that are configured to generate alerts to users when a tracked individual's blood glucose level is out of range and related functionality.

### BACKGROUND

Significant health issues arise when a person's blood glucose level is too high, with people potentially having issues with vomiting or potentially falling into a coma. Additionally, significant health issues may arise when a person's blood glucose level is too low, with such individuals potentially losing consciousness. Thus, many people with diabetes, hypoglycemia, or other diseases that affect blood glucose levels frequently monitor their blood glucose level to ensure that these levels are appropriate.

Continuous glucose monitors are often used by those with diabetes to measure blood glucose levels for tracked individuals, and data from these continuous glucose monitors is often sent to a smart phone of the user (e.g., the wearer, a guardian of the wearer, a healthcare provider of the wearer, etc.) and/or to remote server(s). However, users can often forget to regularly monitor the blood glucose levels when relevant data is merely available in a phone application, and a user may unknowingly continue with what they are doing without realizing that their blood glucose level is poor or very poor and, sometimes, in need of immediate attention. Further, when a user or a tracked individual is not using such a mobile device, away from it, or otherwise disposed, such as sleeping, such notifications may be critical. In cases of outages in cellular service, power outages, and in other similar instances, the phone and other servers may not be capable of obtaining data from continuous glucose monitors, preventing the user from being able to reliably track blood glucose levels. Users are often completely unaware that power has gone out and that alerts are unavailable. Users may also be unable to reliably track blood glucose levels where the phone is out of power, set on do-not-disturb, in a sleep mode, in an airplane mode, etc. Alerts are oftentimes presented in a standard fashion where the alert does not change, and this leads to brain habituation for the user. Additionally, when only audio alerts are generated, a maximum volume for a phone is often insufficient, such as to wake some people from sleep.

Various devices and applications used for tracking blood glucose levels merely generate alerts based on the blood glucose levels alone and do not account for other factors that may be present. By not considering other factors, the devices and applications are less accurate in determining when alerts are necessary, generating more false alarms when no alert is necessary and also generating alerts in a delayed fashion in some instances.

### BRIEF SUMMARY

Embodiments of the present invention contemplate functionality in glucose monitoring devices that enable interaction with external devices for providing alerts, so as to increase the likelihood that a user is properly notified. For example, some example monitoring devices may be configured to connect (e.g., wired or wirelessly) with other devices, such as via smart home integration (e.g., forward smart home integration and/or reverse smart home integration) or other through other techniques. For example, monitoring devices may be configured to cause adjustments in the operation of other external devices, such as in the home, based on data at the monitoring devices. For example, external lights may be adjusted in color, external devices may be turned on or off based on the state of the monitoring devices, adjustments may be made at other external displays, etc., which may be beneficial to effect changes at external devices that are external to the monitoring devices, increasing the likelihood that a user may detect an alert. Monitoring devices may also be configured to cause adjustments in the operation of other external devices, including those outside of the home. For example, monitoring devices may be configured to cause adjustments in the operation of external devices at a workplace of a user, in a vehicle of a user, in a backyard of a user, at a mobile device of a user, and at other locations. Monitoring devices may also be connected to external alert devices (e.g., pucks), and these external alert devices may be positioned on a bed frame or at other locations and may be configured to vibrate to wake up a user sleeping in the bed in the event of an urgent alert.

Additionally, in some embodiments, an external alert device may be provided that may be used to manage the operation of other devices. The device may be configured to act like a controller. The device may receive pulses from monitoring devices described herein. Upon receiving the pulses, the device may determine what other external alert devices need to be adjusted and how the operation of these external alert devices need to be adjusted. For example, the device may receive a pulse, the device may determine which external alert device(s) should be powered on or off, and then the device may cause a signal to be sent to cause the external alert device(s) to be powered on or off.

In some embodiments, through reverse smart home integration or other techniques, adjustments may be made at the monitoring devices based on data received from external devices. For example, when certain external devices are being used in a first room but no external devices are being used in a second room, then a monitoring device may be configured to generate alerts in the first room where devices are being used as this is the room where a user is likely located, and the monitoring device may be configured to refrain from generating alerts in the second room where external devices are not being used.

In some embodiments, example monitoring devices may have a power supply unit that is configured to provide auxiliary power in the event of a power outage. With other devices, power outages may occur and cause the devices to turn off, preventing the devices from providing alerts regarding poor blood glucose levels. Thus, if a power outage occurs during the night, then a user such as a parent could sleep throughout the night without knowing that alerts are unavailable for their child. Even when power outages occur during the day, a user may not contemplate that health alerts are unavailable. Monitoring devices described herein may typically be powered by a first power source, and the power supply unit may provide auxiliary power when power from the first power source becomes unavailable. The power supply unit may provide sufficient auxiliary power to generate an alert at the monitoring device, to send an alert at another device, or to send a notification, text, or the like to a user's smart phone. Thus, when a power outage occurs, a user may be informed of the power outage.

In various example embodiments provided herein, users are able to track blood glucose levels with a monitoring device (e.g., which may be a notification device configured to provide notifications). The monitoring device may present information to the user in a readily understandable manner, with the blood glucose level presented alongside other information. A trend arrow may also be presented that is representative of the rate of change or delta for the blood glucose level. Icons, symbols, emojis, quotes, slang, or other indicators may also be presented that are dependent on the blood glucose levels as well. For example, a unicorn image may be presented on the screen to indicate a “unicorn” blood glucose level, a range of Urgent Low, Low, In Range, High, and Urgent High may be provided to indicate the current blood glucose level for the tracked individuals, an alphanumeric grade may be provided to indicate the current blood glucose level for the tracked individuals, a color may be provided to indicate the current blood glucose level, etc. Information may be presented with emphasis in certain scenarios such as when the blood glucose levels are poor and need attention. Alerts may also be generated, with the alerts being generated by vibration of the monitoring device, a component therein, or a component attached to the device, by audible sounds generated by the monitoring device or a component therein, by flashing certain portions of the screen to provide a visual alert, etc. Alerts may be provided in a randomized manner to prevent brain habituation.

The monitoring device may be configured to obtain data regarding blood glucose levels and/or other information from a number of different data providers (e.g., different manufacturers), allowing the device to be used to track blood glucose levels for multiple tracked individuals, with different data providers being used to track data for the different tracked individuals. In some embodiments, where a tracked individual is using a first tracking device associated with a first manufacturer that uses a first communication protocol and decides to switch to a second tracking device associated with a second manufacturer that uses a second communication protocol that is different from the first communication protocol, the user may still continue to use the monitoring device since the monitoring device may have connectivity to devices made by the first manufacturer, the second manufacturer, and other manufacturers or to data feeds for the different manufacturers.

The monitoring device may be used to track blood glucose data for multiple tracked individuals. This may be beneficial where multiple people within a household have diabetes or otherwise need to track their blood glucose levels. Where the monitoring device is used to present blood glucose levels for multiple tracked individuals, the information associated with each tracked individual may be presented in a certain color to identify the tracked individual or other indications may be provided to indicate that the information presented is associated with a particular tracked individual.

The monitoring device may (via processing in the device and/or remotely located device(s)/server(s)) determine whether or not to alarm based on a trend in the wearer's blood glucose level. In this regard, the monitoring device may take into account whether a wearer's blood glucose level may be adjusting to come back into normal range such that the user does not need to be notified via an alarm (or via certain levels of alarm functionality). For example, if the blood glucose level is going to correct itself and come back into a desirable range, it may not make sense to set off an alarm, such as an alarm that may awaken the user needlessly.

A user (e.g., a tracked individual) may activate a button for some duration of time to generate an alert signal that may be sent to one or more other recipients. This may provide added safety benefits in instances where the tracked individual's blood glucose level is extremely high or extremely low, allowing the tracked individual to alert others to the situation so that the tracked individual may obtain medical assistance. Activation of the button may cause an alert to be generated at another monitoring device or another device such as a user's phone, tablet, etc.

The monitoring device may function as a continuous glucose monitor secondary display, with the monitoring device not being configured to provide any alerts. In other embodiments, the monitoring device may function as a continuous glucose monitor secondary alarm system, with the monitoring device being configured to provide alerts. The monitoring device may be a secondary device that does not directly communicate with the actual on-body medical device, and the monitoring device may accomplish this by, for example, downloading data from the cloud or in other ways.

In an example embodiment, an alert device for management of alerts generated at one or more external devices is provided. The alert device comprises at least one processor and at least one memory device. The at least one memory device comprises computer readable code that, when executed by the at least one processor, is configured to cause the at least one processor to perform various actions. These actions include receiving input data, with the input data comprising blood glucose level data for one or more tracked individuals. The actions also include determining an alert setting for an alert to be generated at an external device of the one or more external devices based on the input data. The alert setting includes at least one of an identification of the external device where the alert is to be generated, a magnitude of the alert, or an alert type, with the alert type being at least one of an audible alert, a haptic alert, or a visual alert. The actions also include generating a signal that causes the alert to be generated at the external device, with the alert having the alert type.

In some embodiments, the input data comprises at least one of time data regarding a time of day, sensor data received from a sensor configured to detect a user in a room, usage data from the one or more external devices, duration data regarding a duration of time that an active alert has been unaddressed, or schedule data regarding a schedule of the user. In some embodiments, the schedule data may comprise data regarding quiet hours for the user when certain alerts are disabled. Additionally, in some embodiments, when duration data indicates that the active alert has been unaddressed for a longer duration, the at least one processor may cause the alert to be generated at the external device with a greater magnitude. Furthermore, in some embodiments, when the duration data indicates that the active alert has been unaddressed for a duration above a first time threshold, the at least one processor may cause the alert to be generated at a different external device based on the duration data relative to when the duration data indicates that the active alert has been unaddressed for a duration above a first time threshold.

In some embodiments, the alert setting may include the alert type, the alert type may be a first alert type when the input data comprises a first dataset, and the alert type may be a second alert type when the input data comprises a second dataset different from the first dataset. Additionally, in some embodiments, the alert setting may include the identification of the external device where the alert is to be generated, the external device may be a first external device when the input data comprises the first dataset, and the external device may be a second external device different from the first external device when the input data comprises the second dataset.

In some embodiments, the external device may be a first external device when the input data comprises a first dataset, and the external device may be a second external device different from the first external device when the input data comprises a second dataset. Furthermore, in some embodiments, the external device may comprise at least one of a monitoring device for presenting blood glucose level information for a tracked individual, a light, a fan, a vibration mechanism, a television, a projection device configured to project an image, a clock, a heating device, a cooling device, an audio device, a device capable of sending a message, or a device capable of making an audio or a video call.

In some embodiments, the blood glucose level data may be received from a blood glucose monitor. In some embodiments, the input data may comprise first blood glucose level data for a first tracked individual and second blood glucose level data for a second tracked individual, the at least one processor may cause the alert to be generated in a first manner when an alert is associated with the first blood glucose level data for the first tracked individual, and the at least one processor may cause the alert to be generated in a second manner different from the first manner when the alert is associated with the second blood glucose level data for the second tracked individual. Additionally, in some embodiments, the at least one processor may cause the alert to be generated at a first external device when an alert is associated with the first blood glucose level data for the first tracked individual, and the at least one processor may cause the alert to be generated at a second external device when the alert is associated with the second blood glucose level data for the second tracked individual.

In some embodiments, the identification of the external device where the alert is to be generated may be determined by determining an urgency level based on the input data. Additionally, in some embodiments, the at least one processor may cause the alert to be generated at a first external device when a first urgency level is present, and the at least one processor causes the alert to be generated at a second external device when a second urgency level greater than the first urgency level is present.

In some embodiments, the at least one processor may cause the alert to be generated at a first external device at a first time of day, and the at least one processor may cause the alert to be generated at a second external device different from the first external device at a second time of day different from the first time of day. In some embodiments, when executed by the at least one processor, the computer readable code may be configured to cause the at least one processor to receive a snooze signal and cause active alerts at the one or more external devices to be snoozed based on the snooze signal.

In some embodiments, the visual alert may comprise a notification on a screen or a projection from a projector, an image on a screen or a projection from a projector, an emoji, flashing lights, activation or deactivation of a light, dimming or brightening of a light, or a change in color on a device. In some embodiments, the haptic alert may result in vibration of one or more of the external devices or activation of a fan to blow air.

In another example embodiment, a system for generation of alerts at one or more external devices is provided. The system comprises the one or more external devices, a blood glucose monitor, and an alert device for management of alerts generated at the one or more external devices. The blood glucose monitor is configured to generate blood glucose level data for one or more tracked individuals. The alert device comprises at least one processor and at least one memory device comprising computer readable code. When executed by the at least one processor, the computer readable code is configured to cause the at least one processor to receive input data, with the input data comprising blood glucose level data received from the blood glucose monitor. When executed by the at least one processor, the computer readable code is also configured to cause the at least one processor to determine an alert setting for an alert to be generated at an external device of the one or more external devices based on the input data. The alert setting includes at least one of an identification of the external device where the alert is to be generated, a magnitude of the alert, or an alert type, with the alert type being at least one of an audible alert, a haptic alert, or a visual alert. When executed by the at least one processor, the computer readable code is also configured to cause the at least one processor to generate a signal that causes the alert to be generated at the external device, with the alert having the alert type.

In some embodiments, the input data comprises at least one of time data regarding a time of day, sensor data received from a sensor configured to detect a user in a room, usage data from the one or more external devices, duration data regarding a duration of time that an active alert has been unaddressed, or schedule data regarding a schedule of the user. Additionally, in some embodiments, the external device may comprise at least one of a monitoring device for presenting blood glucose level information for a tracked individual, a light, a fan, a vibration mechanism, a television, a projection device configured to project an image, a clock, a heating device, a cooling device, an audio device, a device capable of sending a message, or a device capable of making an audio or a video call. In some embodiments, the input data may comprise first blood glucose level data for a first tracked individual and second blood glucose level data for a second tracked individual, the at least one processor causes the alert to be generated in a first manner when an alert is associated with the first blood glucose level data for the first tracked individual, and the at least one processor causes the alert to be generated in a second manner different from the first manner when the alert is associated with the second blood glucose level data for the second tracked individual.

In another example embodiment, a method for generation of alerts at one or more external devices is provided. The method comprises receiving input data, and the input data comprises blood glucose level data received from the blood glucose monitor. The method also comprises determining an alert setting for an alert to be generated at an external device of the one or more external devices based on the input data, and the alert setting includes at least one of an identification of the external device where the alert is to be generated, a magnitude of the alert, and an alert type, with the alert type being at least one of an audible alert, a haptic alert, or a visual alert. The method also comprises generating a signal that causes the alert to be generated at the external device, with the alert having the alert type.

In some embodiments, the input data comprises at least one of time data regarding a time of day, sensor data received from a sensor configured to detect a user in a room, usage data from the one or more external devices, duration data regarding a duration of time that an active alert has been unaddressed, or schedule data regarding a schedule of the user. Additionally, in some embodiments, the external device may comprise at least one of a monitoring device for presenting blood glucose level information for a tracked individual, a light, a fan, a vibration mechanism, a television, a projection device configured to project an image, a clock, a heating device, a cooling device, an audio device, a device capable of sending a message, or a device capable of making an audio or a video call.

### BRIEF DESCRIPTION OF THE DRAWINGS

Reference will now be made to the accompanying drawings, which are not necessarily drawn to scale, and wherein:

FIG. 1 is a front, perspective view illustrating an example monitoring device, in accordance with some embodiments discussed herein;

FIG. 2 is a rear, perspective view illustrating an example monitoring device, in accordance with some embodiments discussed herein;

FIG. 3 is a front view illustrating an example monitoring device, in accordance with some embodiments discussed herein;

FIG. 4 is a top view illustrating an example monitoring device, in accordance with some embodiments discussed herein;

FIG. 5 is a right side view illustrating an example monitoring device, in accordance with some embodiments discussed herein;

FIG. 6 is a left side view illustrating an example monitoring device, in accordance with some embodiments discussed herein;

FIG. 7 is a block diagram illustrating various components of a system for operating a monitoring device, in accordance with some embodiments discussed herein;

FIG. 8 is a schematic view illustrating various devices shown within different rooms within a home and how a monitoring device may operate through, for example, forward smart home integration to make changes to other devices in the home or elsewhere based on data at the monitoring device and how the monitoring device may operate through, for example, reverse smart home integration to adjust operation of the monitoring device based on data from other devices within the home or elsewhere, in accordance with some embodiments discussed herein;

FIG. 9A is a perspective view illustrating an example projection device that is external to the monitoring device, in accordance with some embodiments discussed herein;

FIG. 9B is a rear view of the projection device of FIG. 9A, in accordance with some embodiments discussed herein;

FIG. 10 is a perspective view of an example connector device that may be used to connect a monitoring device and another external device, in accordance with some embodiments discussed herein;

FIG. 11A includes schematic views of three different example external alert devices that may be vibrated to alert a user, in accordance with some embodiments discussed herein;

FIG. 11B is a schematic view illustrating the external alert devices of FIG. 11A attached to a bed frame, in accordance with some embodiments discussed herein;

FIG. 12 is a schematic view of another external alert device that may be used to control or manage the operation of other devices, in accordance with some embodiments discussed herein;

FIG. 13 is a front, perspective view illustrating an example monitoring device that shows an emoji representation indicative of a tracked individual's current blood glucose level, in accordance with some embodiments discussed herein;

FIG. 14 is a front, perspective view illustrating an example monitoring device that shows an enlarged blood glucose level and a trend arrow, in accordance with some embodiments discussed herein;

FIG. 15 is a front, perspective view illustrating an example monitoring device that is configured to present information for two different tracked individuals, in accordance with some embodiments discussed herein;

FIG. 16 is a front, perspective view illustrating an example monitoring device that includes the blood glucose level, the change in that level over a time interval, and a trend arrow, in accordance with some embodiments discussed herein;

FIG. 17 illustrates a continuous glucose monitor attached on an arm of a tracked individual, in accordance with some embodiments discussed herein;

FIG. 18 illustrates graphs showing blood glucose levels as a function of time with blood glucose levels approaching a lower threshold of a normal or desired zone for blood glucose levels, in accordance with some embodiments discussed herein;

FIG. 19 illustrates an example insulin pump and a smart insulin pen, in accordance with some embodiments discussed herein;

FIG. 20 is a flow chart illustrating an example method for adjusting an external alert device based on blood glucose level data, in accordance with some embodiments discussed herein;

FIG. 21 is a flow chart illustrating an example method for adjusting operation of the monitoring device based on data from other devices within the home or elsewhere, in accordance with some embodiments discussed herein;

FIG. 22 is a flow chart illustrating an example method for generating alerts regarding a lack of availability for blood glucose level data or other data, in accordance with some embodiments discussed herein;

FIG. 23 illustrates an example flow chart with a method for presenting blood glucose level information for a tracked individual, in accordance with some embodiments discussed herein;

FIG. 24 illustrates an example flow chart with a method for presenting blood glucose level information for a tracked individual, in accordance with some embodiments discussed herein;

FIG. 25 illustrates an example flow chart with a method for presenting blood glucose level information for a tracked individual, in accordance with some embodiments discussed herein; and

FIG. 26 illustrates an example flow chart with a method for generation and snoozing of alerts at one or more external devices, in accordance with some embodiments discussed herein.

### DETAILED DESCRIPTION

Example embodiments now will be described more fully hereinafter with reference to the accompanying drawings, in which some, but not all embodiments are shown.

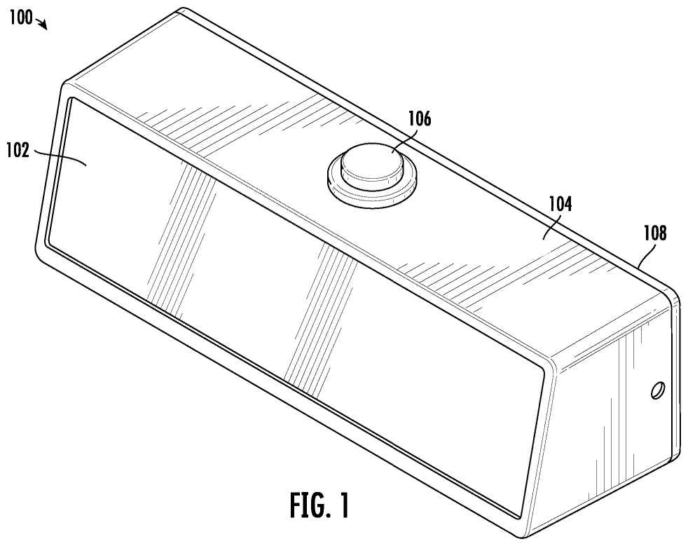

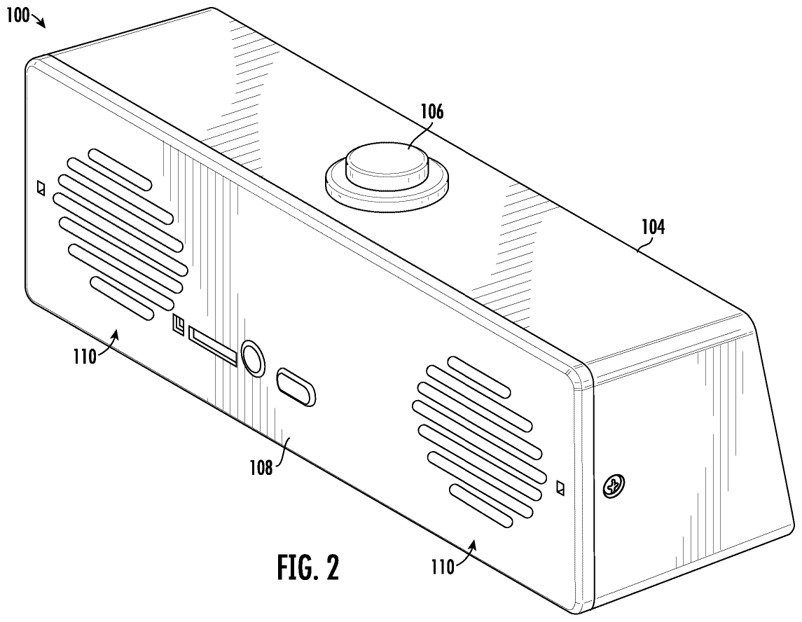

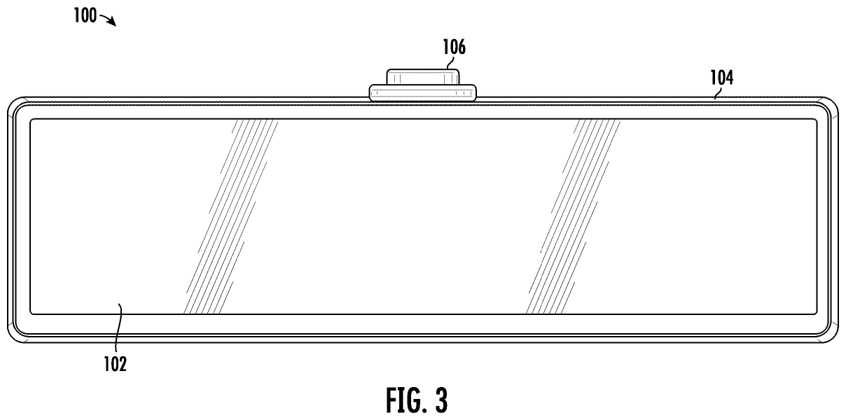

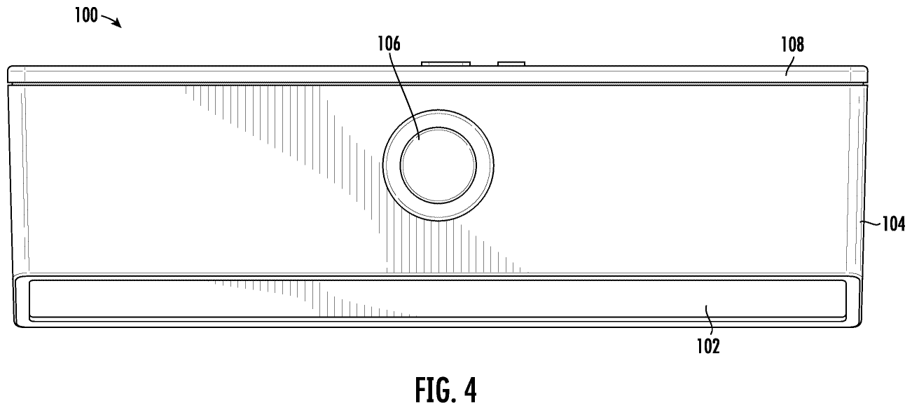

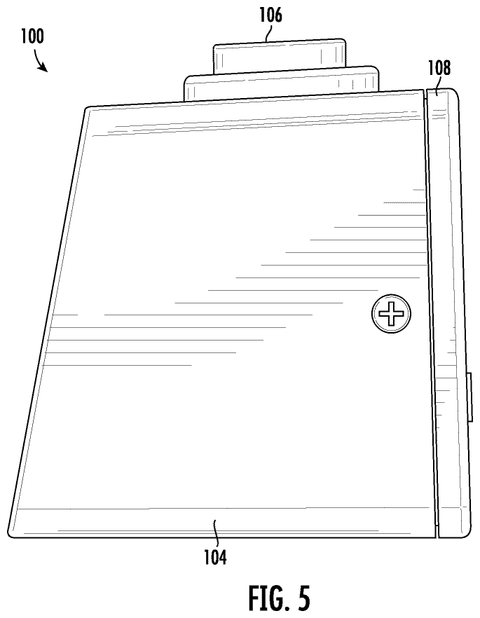

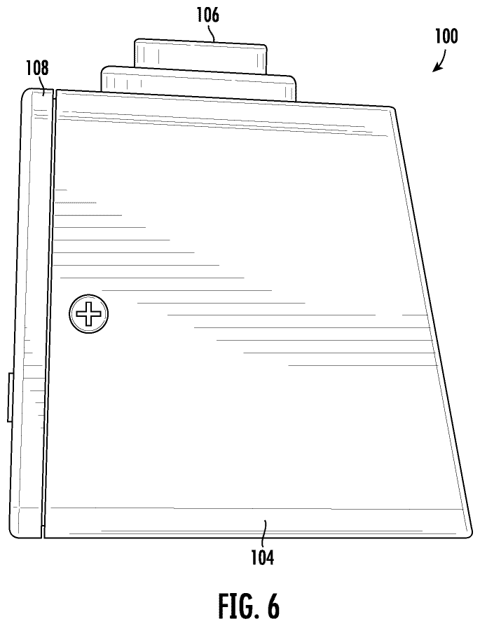

One example monitoring device is illustrated in FIGS. 1-6, with FIG. 1 being a front, perspective view, with FIG. 2 being a rear, perspective view, with FIG. 3 being a front view, with FIG. 4 being a top view, with FIG. 5 being a right side view, and with FIG. 6 being a left side view. The monitoring device 100 may be a dedicated and standalone hardware device that operates separately from any tracking device itself and separate from any phone (or other user device). The monitoring device 100 may display blood glucose levels for one or more tracked individuals. The monitoring device 100 comprises a screen 102 where information may be presented, and the information that may be presented on the screen 102 is described in greater detail herein. While the monitoring device 100 comprises a screen 102, other monitoring devices may be provided without a screen, and information about a tracked individual's blood glucose levels or health status may be presented in other ways. For example, this information may be presented via a buzzer, an indicator light, a speaker, a projector, etc.

The monitoring device 100 also comprises an outer shell 104 that protects that inner contents of the monitoring device 100, with electronics positioned within the outer shell 104. A back cover 108 may be removably attachable to the outer shell 104, with the back cover 108 extending all the way along one side of the monitoring device 100. The back cover 108 may be removably attachable so that the inner contents of the monitoring device 100 may be added, removed, cleaned, or otherwise maintained. Additionally, as illustrated in FIG. 2, the back cover 108 may define openings 110 to provide ventilation for the inner contents of the monitoring device 100. The openings 110 may also be configured to allow sound to easily exit the monitoring device 100 through the openings 110.

A button 106 is also included, with the button 106 extending upwardly through the outer shell 104. The button 106 may be pushed to snooze or turn off alerts and to input other information to the monitoring device 100. While only one button 106 is illustrated for the monitoring device 100, other embodiments may include multiple buttons. In some embodiments, button 106 or another button may be pressed to cause multiple monitoring devices and/or other devices to be snoozed at one time (as used herein “snooze” may refer to causing the alert to be temporarily disabled and/or canceled). In some embodiments, pressing of a button on the monitoring device 100 in a certain manner (e.g., for a certain duration of time, in a certain tapping pattern, etc.) may cause snoozing of alerts being generated at that monitoring device 100 and/or at other devices (e.g., at another monitoring device, at cell phone of a parent, friend, etc., or at a tablet, a computer, etc.). However, in some embodiments, pressing the button on the monitoring device 100 may cause snoozing of alerts only on the monitoring device 100 itself or only on one other external device.

In some embodiments, the monitoring device 100 may be configured to provide remote alerts. With the remote alerts, the button 106 and other buttons described herein may be activated for an extended period of time, and this may cause the monitoring device to generate an alert signal that may be sent to another device or to indicate that there is a medical emergency. For example, the user (e.g., a tracked individual) may press the button 106 and hold down on the button 106 for about 1 second or more, about 3 seconds or more, about 5 seconds or more, or for another threshold period of time. However, in some embodiments, a button may simply be activated to generate the alert signal. Where this is the case, a cover may be added in some embodiments over the button to prevent the button from inadvertently being pressed.

The ability to provide remote alerts provides additional safety benefits for the monitoring device. If a tracked individual's blood glucose level gets really low, the blood glucose level may become so dangerous that the tracked individual may lose consciousness. When the person is in this state, functioning at all may be difficult. If the person is in this state, the person may still be able to press and hold the button 106 or issue a voice command. By pressing the button 106 or by issuing a voice command, an alert signal may be sent to the monitoring device 100 or another similar monitoring device, to a user device (e.g., a phone, a receiver, tablet, etc.) of another person or persons so that the individual being tracked may get assistance. In some embodiments, the monitoring device 100 may send a command using the internet that gets routed to another device, and this may be done through message queuing telemetry transport (MQTT) or other known internet messaging protocols. Once the alert signal is received at the other device, the recipient may receive a unique alarm that obtains the attention of the recipient. Additionally, other important information may be presented to the recipient such as the location of the tracked individual that is in danger, the name of the tracked individual, the name or some identification of the device, the blood glucose level for the tracked individual, the potential medical risks to the tracked individual (e.g., low blood glucose level, potential loss of consciousness, among others). However, further information may be presented, different information may be presented, or none of this information may be presented to the recipient in some embodiments. In some embodiments, signals may be sent to multiple receiving devices (e.g., to a loved one and to local authorities to obtain medical assistance). The ability to generate a remote alert also provides an extra layer of safety in the event an alert is not automatically generated for some reason. For example, readings from tracking devices may, at times, be inaccurate due to a variety of causes, such as dehydration. Despite the fact that an actual blood glucose level may be at a dangerous level, inaccurate readings may prevent alerts from being triggered because the readings may errantly indicate the tracked individual is in a normal range. Most tracked individuals are capable of feeling when their blood glucose is low and triggering the remote alert for assistance. The remote alert to a user's device may be the only alert that the user receives.

In some embodiments, the monitoring device may be configured to receive out-of-range indications from the tracking device that the blood glucose level either falls inside or outside of a desired range, and these out-of-range indications may not include a specific blood glucose level. These out-of-range indications may be sent at a greater frequency than other indications that include a specific blood glucose level. For example, indications having specific blood glucose level may be sent every five minutes, but out-of-range indications may be sent every minute and/or whenever they occur. The use of both out-of-range indications and indications with specific blood glucose level may be beneficial to optimally use the information made available by tracking devices. In this regard, some tracking devices are configured to track whether blood glucose levels are out-of-range at a higher frequency (e.g., once a minute) than the frequency at which the tracking devices track specific blood glucose levels (e.g., once every five minutes). In some embodiments, if an indication is sent indicating that the blood glucose level is out of range, then the monitoring device or another device may be configured to cause another indication to be generated indicating the specific blood glucose level for the tracked individual-however, if an indication is sent indicating that the blood glucose level is within range, then no additional indication may be sent regarding the specific blood glucose level at that time.

Additionally or alternatively, the monitoring device 100 may include other user interfaces so that the user may input data or cause changes in the operation of the monitoring device 100. In some embodiments, the monitoring device 100 may be connected to a phone or another user device through an application on the phone or the user device, and the user may input data or cause changes in the operation of the monitoring device 100 using the application on the phone. For example, the user may snooze an alert for one or more monitoring device s at the user's phone. Where this is the case, the user may, for example, be a parent who wishes to monitor blood glucose levels for two of the parent's children—when multiple monitoring device s have their alarms going off, the parent may press a button to snooze one of the alerts or to snooze multiple active alerts.

Additionally, in some embodiments, the monitoring device 100 may be configured to provide vibration alerts. With the vibration alerts, the monitoring device 100 or a vibration device within the monitoring device 100 or externally connected to the monitoring device 100 may vibrate to get the attention of the user. In some embodiments, vibration alerts may be provided with randomized audio and vibration patterns to help prevent brain habituation. For example, the alerts may be provided in a first pattern on a first occasion, a second pattern on a second occasion, and a third pattern on a third occasion, with each of the patterns being different from each other. In some embodiments, the audio and/or vibration pattern may be randomly generated on each occasion or selected from a group of available patterns on each occasion, and the group of available patterns may have two or more patterns, three or more patterns, five or more patterns, ten or more patterns, etc. Where audio and/or vibration patterns are randomly generated, the length of any audio or vibration may be adjusted, the pitch or magnitude of audio or vibration may be adjusted, the duration of silence between audio notes or periods with vibration may be adjusted. Even within a single pattern, these parameters may be changed (e.g., audio or vibration may be generated for 5 seconds, pause for 1 second, and may be generated again for only 2 seconds). Patterns may be different from each other in terms of the magnitude of vibration, the duration of time that vibration occurs, the duration of time that vibration does not occur, the frequency of vibration, the frequency of instances where vibration does not occur, the noise level of any audio that is generated, the audio source, the duration of time that audio is played, the duration of time that audio is not played, the frequency at which audio plays, the frequency of instances that audio does not play, etc.

In some embodiments, the alert volume may be incremented so that the volume becomes incrementally louder over time. Alternatively, the first instance of an alert may simply trigger a vibration alert, and subsequent instances of an alert may trigger an audio alert and/or a vibration alert, with the alerts escalating in magnitude. Thus, if the user fails to address the alerts that are generated, the alerts may continue to get louder or may otherwise have a greater magnitude until the user addresses the issue or disables the alerts to indicate that the issue has been resolved. In some embodiments, the alerts may become louder or may have a greater magnitude after each minute that the alert is not snoozed, but the alerts may increase in volume or magnitude in different time intervals (e.g., once every 5 seconds, once every 15 seconds, once every 30 seconds, once every 2 minutes, once every 5 minutes, etc.).

In some embodiments, the user may receive an emergency communication if the user(s) do not address an alert within a specified time. This emergency communication may be a call or a text to a designated number, or the emergency communication may be an email sent to a designated email address. The user may be able to adjust the settings in some embodiments to enable or disable emergency communications, to enable or disable emergency communications at certain time windows, to change the time before any emergency communication is made, or to automate the emergency communication if an alert has not been snoozed within a certain time. In some embodiments, the monitoring device may be configured to make an emergency communication to another device if the alert has not been snoozed within a certain time, and this emergency communication may be provided in the form of a phone call, a text message, an email, a notification, etc.

In some embodiments, snoozing alerts may be generated on other devices that are remote from the monitoring device 100. For example, alerts may be generated on a smart phone, a receiver, a tablet, a computer, or another device, with signals being sent via a wireless connection in some embodiments. Signals may be sent via low range wireless communication approaches such as Bluetooth, Bluetooth low energy (BLE), Wi-Fi, etc. However, signals may be sent using other wireless communication technologies. In some embodiments, signals may be sent via wired connections between the monitoring device and other devices. For example, the monitoring device may be connected via a wired connection to a smart phone, a receiver, a tablet, a computer, etc., and a snoozing alert may be generated at the tablet or the computer.

In some embodiments, alerts may be provided to indicate when no data is available or where limited data is available. This may, for example, occur when local outages occur, when the continuous glucose monitor or another tracking device is out of power or is disconnected, when the communication channel for data is disconnected (e.g., when Bluetooth, Wi-Fi, etc. are disconnected), or when other issues arise that cause data to be unavailable. In instances where the communication channel for data is disconnected, data may still continue to be collected by the tracking device so that the data may be uploaded to a monitoring device or to the smart phone of the user.

In some embodiments, distributed outage detection may be provided to users to indicate when large-scale outages are occurring. For example, when one or more products or services provided by a third-party manufacturer (e.g., Dexcom®, FreeStyle Libre®, etc.) are down, indications may be provided to users to inform them of the issue. These indications may be provided at the monitoring device s, at another user device, or at another location. In some embodiments, the indications associated with distributed outage detection may be provided at a website that the user may access using his or her user device, and the indications may provide detailed information regarding the problem and the status of any troubleshooting. These distributed outage detection indications may inform the user that the problems are not related to any hardware or software issues with his or her particular devices, giving the user clarity regarding the problem as well as peace of mind. By providing distributed outage detection indications at a website, the traffic level at the website may be increased, which may lead to increased visibility for the products and which may potentially lead to increased product sales.

Further details regarding monitoring devices may be found in U.S. patent application Ser. No. 18/943,187, entitled “Glucose Monitoring Alarm Device and Related Functionality”, which is incorporated by reference herein for all purposes.

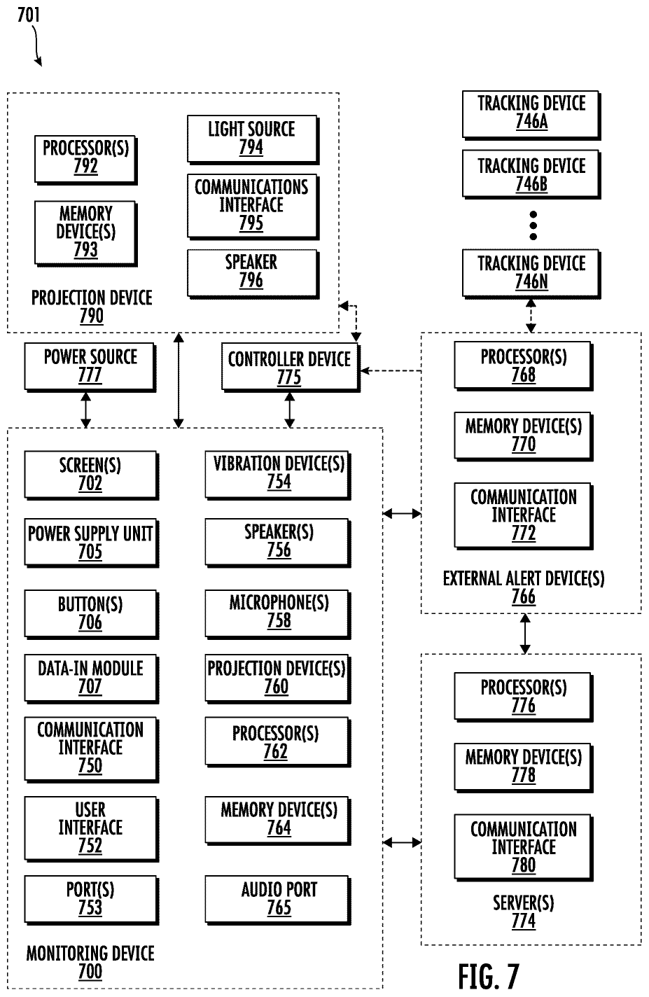

FIG. 7 illustrates a block diagram with various components of a system 701 for operating the monitoring device 700. The system 701 may comprise a monitoring device 700, a plurality of tracking devices 746A-746N, one or more external alert devices 766, one or more servers 774, a power source 777, and a projection device 790. Tracking devices 746A-746N may comprise at least one of a blood glucose monitor (e.g., a continuous blood glucose monitor), a sensor associated with an insulin pump, or an insulin pen (which may be a smart insulin pen that is configured to track insulin doses), but the tracking devices 746A-746N may take some other form.

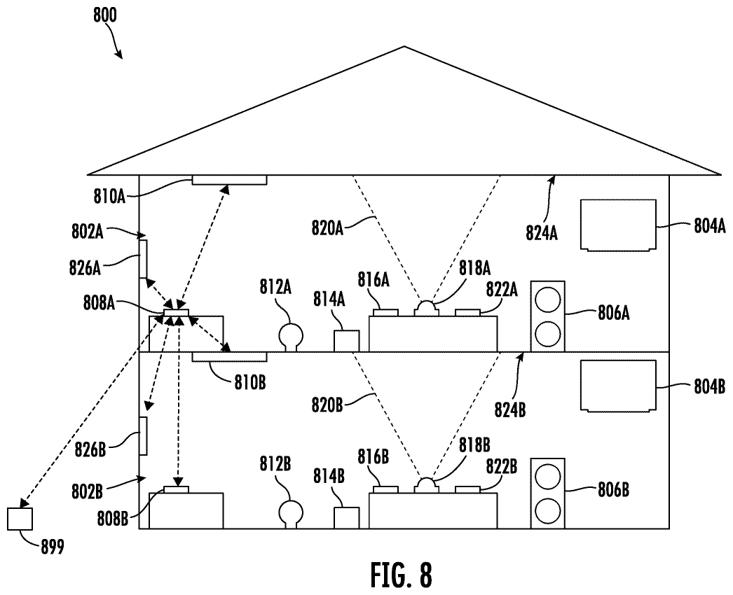

External alert device(s) 766 are also included in the system 701, and the external alert device(s) 766 are devices external to the monitoring device 700 that are configured to alert the user regarding issues. Various external alert device(s) 766 may be used, and various examples are described in the discussion of FIG. 8. The external alert device(s) 766 may include one or more processors 768, one or more memory devices 770, and a communication interface 772. The external alert device(s) 766 may comprise at least one of a computer, a cell phone (e.g., a smart phone), a tablet, a receiver, a display, or the like in some embodiments. Additionally or alternatively, external alert device(s) 766 may comprise at least one of a light, a fan, a vibration mechanism, a television, a projection device configured to project an image, a clock, a heating device, a cooling device, or an audio device, a device capable of sending a message, or a device capable of making an audio or a video call. However, other external alert device(s) 766 may be used. External alert device(s) 766 may be located in a user's home, but these device(s) 766 may be located in other places as well. For example, external alert device(s) 766 may be provided in a user's vehicle, in a user's workplace, in a user's backyard, etc. In some embodiments, data may be driven to other external alert device(s) 766 for use with other software or applications like Nightscout™, Gluroo™, etc. The system 701 may also include one or more servers 774. The server(s) 774 may include one or more processors 776, one or more memory devices 778, and a communication interface 780. These components of the external alert device(s) 766 and the server(s) 774 are described in greater detail herein.

The power source 777 may provide power for the monitoring device 700 during normal operation of the monitoring device 700. The power source 777 may be a power outlet located at a wall or at another location, but other power sources 777 are contemplated herein.

The monitoring device 700 also includes a power supply unit 705. The power supply unit 705 may be configured to supply sufficient auxiliary power to generate an alert when power from the power source 777 becomes unavailable. This may be particularly beneficial where a user loses power overnight. If power is lost overnight and the power supply unit 705 is not used, the user may remain sleeping without realizing that alerts from the device are unavailable. With the power supply unit 705 being available, the auxiliary power from the power supply unit 705 may provide sufficient power to enable the power loss to be detected and to generate an alert to the user. Thus, the user may be woken up upon receiving the alert. However, in some embodiments, this functionality may optionally be turned off by the user if the user chooses not to receive such power outage alerts.

The alert may be an audio alert, a text alert sent to a remote device, or a notification sent to a remote device, with the remote device being the external alert device or another external device. In some embodiments, no-power alerts may be sent from the monitoring device 700 to other devices (e.g., cell phones, smart phones, tablets, etc.) using cellular data. The power supply unit 705 may comprise a battery and/or a capacitor, but the power supply unit 705 may comprise other components. While the power supply unit 705 is only illustrated in the monitoring device 700, other similar power supply units may be implemented in other devices (e.g., projection device 790, tracking device 746A, external alert device(s) 766, etc.), thereby enabling the devices to generate similar no-power alerts.

The monitoring device 700 includes a data-in module 707. The data-in module 707 may be configured to receive external data from an external source, and the external source may be located away from the monitoring device 700. The external data received at the data-in module 707 may comprise data related to an insulin level for the tracked individual, other health data for the individual (sleep amounts, heart rate, steps, distance moved, or the like), but other external data may be received at the data-in module 707 for use at the monitoring device 700. The insulin levels for a tracked individual may be tracked by assessing how much insulin the tracked individual has within his or her system at any given time, by assessing the amount of insulin consumed within a particular time period (e.g., from an insulin pump, an insulin pen, or the like), or through another alternative approach. Tracking the insulin levels and/or other data may be beneficial to enable more accurate alerts to be generated. For example, when the insulin levels and blood glucose levels together are indicative of an issue, then an alert may be generated. However, there may be other situations where a blood glucose level standing alone appears to fall outside of range, but a determination may be made that no alert is needed given the insulin level at that time-thus, a monitoring device may avoid a false alarm where an alert is triggered unnecessarily. Data may be received at the data-in module 707 from various external devices (e.g., smart phones, tracking devices, sensors, etc.). The data-in module 707 may be configured to receive other types of data as well such as historical data about a particular user, and this data may be used in making determinations of whether or not to generate an alert and determinations of the type of alert to be generated. Furthermore, data regarding the derivatives of the blood glucose levels as a function of time may be considered in making determinations regarding alerts.

The data-in module 707 may enable more complex information and/or expanded displays to be presented on the monitoring device 700 and may enable more complex determinations to be made at the monitoring device 700 about how the operation of the monitoring device 700 or other devices should be adjusted. While the data-in module 707 is provided as a separate component from the communications interface 750 in the illustrated embodiment, the data-in module 707 may be included as part of the communications interface 750 in other embodiments. In some embodiments, the data-in module 707 may be omitted.

The data-in module 707 may be used to obtain data to allow for a determination of blood glucose levels from alternate sources or from non-blood glucose data. In some embodiments, the data-in module 707 may comprise an adapter allowing Bluetooth-in data feeds from a mobile application so that a user may make the monitoring device 700 function in “camping/off grid” mode where there is no Wi-Fi required and the monitoring device 700 receives blood glucose level values directly from the user's smart phone. The data-in module 707 may also be used to programmatically download insulin pump data or data from other sources such as from Apple Health™, other health applications, or other applications on a user's smartphone. By receiving these various types of data in the data-in module 707, the type of information provided on the display of the monitoring device 700 may be expanded.

Modules disclosed herein (e.g., the data-in module 707) may refer to portions of computer program code and/or the processor/special circuitry that implements that functionality. Modules may be provided in the form of one or more processors in some embodiments, or modules may be provided as a subcomponent within one or more processors. Modules may be hardware and/or software implemented to substantially perform the particular functions discussed. Moreover, the modules may be combined together within the disclosure, or divided into additional modules based on the particular function desired.

The monitoring device 700 includes one or more ports 753 and an audio port 765, but these ports may be omitted in other embodiments. These port(s) 753 may include USB-C ports in some embodiments. Where USB-C ports are used, external devices may be easily set-up by connecting a USB-C cable to the ports. By using USB-C ports, accessories may be easily detectable for automatic setup and use because the accessory and main device may communicate relevant data to each other through the USB-connection. For example, an external display (e.g., a television or a computer monitor) may be connected to the monitoring device 700 using the USB-C port. The USB-C connection between the display and the monitoring device 700 may allow the display to communicate data to the monitoring device 700 to inform the monitoring device 700 that it is connected to a display and/or particular properties of the display. Based on this data, the monitoring device 700 may immediately begin sending display data over the USB-C connection for immediate functionality without any user setup other than plugging it in. Other devices connected to the monitoring device 700 may function similarly. Some devices may allow further configuration if desired and some devices may require additional steps for configuration, but the use of USB-C ports enables more rapid set up of external devices. In some embodiments, a mobile application may be used, and this mobile application may detect the accessories that are plugged in to be able to display the user with additional or alternative settings and options in the application.

USB-C ports may provide more universal compatibility with other devices such as laptops, smartphones, tablets, and the like and may reduce the need for multiple types of cables. As a result of this more universal compatibility, monitoring device 700 may be provided with a reduced number of ports 753, and the monitoring device 700 may even be provided with just one port 753 in some embodiments. By doing so, the cost of manufacturing the monitoring device 700 may be reduced, allowing for greater cost-efficiency during manufacturing. USB-C ports may have high data transfer speeds (e.g., above about 480 Megabits per second (Mbps), above about 5 Gigabits per second (Gbps), above about 10 Gbps, above about 20 Gbps, above about 40 Gbps, or even above about 80 Gbps) and may be capable of delivering a high amount of power (e.g., about 240 Watts or more). The use of USB-C ports may help reduce the number of cables needed to provide power and/or to transfer data, and the USB-C ports may allow for a one-cable solution for transferring power and various types of data in some embodiments. Thus, the inclusion of USB-C ports may make the monitoring device 700 easier to use. While the port(s) 753 may be USB-C ports in some embodiments, the port(s) 753 may be USB ports or other types of ports in other embodiments. For example, ports described herein may be provided in the form of a USB-A port, a micro-USB port, an audio jack port, a 110 volt port, a 220 volt port, or in another form. An audio jack port may be configured to provide additional or diverted audio. In some embodiments, ports may be configured to deliver both power and data regarding communications, but ports may only be configured to deliver power or data regarding communications in other embodiments.

The connections illustrated by arrows in FIG. 7 may be formed in various ways. In some embodiments, some of these connections may be formed using wired connections. For example, the connector device 1000 described in greater detail elsewhere herein may be used to connect the monitoring device 700 to the projection device 790, to the power source 777, to the external alert device(s) 766, or to the server(s) 774. However, other wired connections may be made without using the connector device 1000 in other embodiments. In some embodiments, some of the connections may be formed using wireless connections as described in greater detail elsewhere herein.

The monitoring device 700 also comprises an audio port 765. The audio port 765 may be configured to allow for additional audio or diverted audio. For example, an external speaker may be connected through a wired connection using the audio port 765, and this may enable sound to be generated at both the speaker(s) 756 within the monitoring device 700 and at the external speaker. In some embodiments, the external speaker may be configured to generate sounds at higher decibel levels than the speaker(s) 756 in the monitoring device 700, so playing audio at the external speaker may increase the likelihood of a user noticing an alert. Additionally, in some embodiments, the external speaker may be positioned at another location away from the monitoring device 700, such as in a different part of a room. For example, a monitoring device may be positioned on one nightstand on one side of a bed, and an external speaker may be positioned on another nightstand at the other side of the bed, thereby allowing both spouses to receive audio on their respective side of the bed. In some embodiments, a user may have wired or wireless headphones connected in the audio port 765, thereby allowing alerts to be detected by one spouse or roommate in his or her headphones without waking or otherwise disturbing the other spouse or roommate. However, wireless headphones or speakers may be connected to the monitoring device 700 in other ways, such as through the use of the communication interface 750. Additionally, in some embodiments, the external speaker may be positioned in a different room or even outside of a home (e.g., outdoor speakers on a porch). The use of an external speaker may increase the likelihood of a user noticing an alert when the user is located away from the monitoring device 700 itself. While external speakers may be connected via an audio port 765, external speakers may additionally or alternatively be connected wirelessly through the use of the communication interface 750 in some embodiments. However, the audio port 765 may be omitted in some embodiments.

In some embodiments, the monitoring device 700 and other monitoring devices described herein may be configured to be used for multiple tracked individuals. Where this is the case, monitoring devices may be configured to generate a unique alert for each tracked individual. For example, the monitoring device may be configured to provide a first audio alert when a blood glucose level of a first tracked individual is out of range, and the monitoring device may be configured to provide a second audio alert different from the first audio alert when the blood glucose level of a second tracked individual is out of range. In other embodiments, the monitoring device may present different visual alerts for the tracked individual (one with a first color, emoji, flashing pattern, etc., and the other with a distinctive second color, emoji, flashing pattern, etc.). In some embodiments, the monitoring device may generate alerts of different types for each tracked individual (e.g., audio alert for one, vibratory alerts or visual alerts for the other).

Additionally, in some embodiments, the monitoring device 700 and other monitoring devices described herein may be configured to cause an alert to be generated at different devices for different tracked individuals. For example, a first alert may be generated at an external device connected to the monitoring device 700 via the audio port 765, and a second alert may be generated at the monitoring device 700 itself. Alternatively, the monitoring device 700 may refrain from generating alerts at all, the monitoring device 700 may be configured to cause a first external device to generate an alert for a first tracked individual when appropriate, and the monitoring device 700 may be configured to cause a second external device to generate an alert for a second tracked individual when appropriate. These are merely examples, and the monitoring devices described herein may be configured to be used in conjunction with more than two tracked individuals, and the monitoring devices may be configured to cause alerts to be generated at multiple devices at the same time for a particular tracked individual.

The monitoring device 700 includes a display in the form of one or more screens 702. The screen(s) 702 may be configured to present images and may include or otherwise be in communication with button(s) 706 and/or a user interface 752 configured to receive input from a user. The screen(s) 702 may be, for example, a conventional LCD (liquid crystal display), a touch screen display, or any other suitable display known in the art upon which images may be displayed. However, in some embodiments, no screen is provided. Where a monitoring device is provided without any screen, a power light or some other indicator may be included to indicate that the monitoring device is on or to indicate a state of the monitoring device. Alternatively, a monitoring device without a screen may have a blinking light where the blink indicates that the monitoring device is operating correctly, and the color of the light may change based on the blood glucose level.

The monitoring device 700 may also include a user interface 752. The user interface 752 may include, for example, a keyboard, keypad, function keys, buttons, a mouse, a scrolling device, input/output ports, a touch screen, or any other mechanism by which a user may interface with the system. The monitoring device 700 may also include one or more buttons 706. The button(s) 706 may be in the form of a push button or another actuatable mechanism where the user(s) may activate the button(s) 706 to input information for use at the monitoring device 700. For example, the button(s) 706 may be activated to snooze an alarm on the monitoring device 700 or to input other information. The monitoring device 700 also includes a communication interface 750. The communication interface 750 is discussed in greater detail herein.

The monitoring device 700 may also include one or more vibration devices 754. The vibration device(s) 754 may be a buzzer or some other device that generates vibration within the monitoring device 700 or external to the monitoring device 700. Vibration device(s) 754 may be activated to generate vibration for an alarm. The vibration device(s) 754 may be configured to generate vibration in differing or even random ways to prevent users from experiencing brain habituation.

The monitoring device 700 comprises one or more speakers 756. The speaker(s) 756 may be used to provide audio alerts. Additionally, the speaker(s) 756 may provide blood glucose levels in audio form for those with visual impairments, and the blood glucose levels may be converted from text or numbers to audio that is generated at the speaker(s) 756. The audio alerts may be in the form of generic sounds (e.g., clapping), themed sounds (e.g., happy Christmas music), or the like. In some embodiments, computer readable code on the monitoring device 700 may cause similar audio alerts to be generated at other external alert devices in some embodiments. However, the speaker(s) 756 may be omitted in some embodiments.

The monitoring device 700 also includes one or more microphones 758. The microphone(s) 758 may be configured to receive voice commands from the user(s) or others. For example, the voice commands may be used to change the settings for the monitoring device 700 so that the brightness level is changed, so that user information is changed, so that the color scheme is changed, to activate an emergency notification to a remote device, and to make other changes. However, in some embodiments, no microphone is included in the monitoring device 700.

The monitoring device 700 also includes one or more projection devices 760. The projection device(s) 760 may be configured to project images towards a ceiling or towards another surface. The projection device(s) 760 may generate a single color, an image, a symbol, a number, or the like, and the material that is generated may be dependent on the blood glucose level of the tracked individual. For example, the projection device(s) 760 may be configured to generate themed images (e.g., northern lights theme, Christmas theme, or the like).

In some embodiments, a projection device 790 may be provided external to the monitoring device 700. The projection device 790 may include one or more processors 792, one or more memory devices 793, a light source 794, a communications interface 795, and a speaker 796. However, the projection device 790 may include other components in other embodiments. The projection device 790 may be configured to project images towards a ceiling or towards another surface with images being generated using light from the light source 794. The projection device 790 may generate a single color, an image, a symbol, a number, or the like, and the material that is generated may be dependent on the blood glucose level of the tracked individual. For example the projection device(s) 790 may be configured to generate themed images (e.g., northern lights theme, Christmas theme, or the like). While the system 701 is shown with a projection device 790 external to the monitoring device 700 and also projection device(s) 760 within the monitoring device 700, projection device 790 and/or projection device(s) 760 may be omitted in other embodiments.

The processor(s) 792 may be configured to perform a variety of functions, and the processor(s) 792 may execute operations or instructions stored at the memory device(s) 793 or at another location. Processor(s) 792 may cause images to be generated using the projection device 790, the light source 794 to be turned on and off, and/or the speaker 796 to generate sounds. However, the processor(s) 792 may be configured to perform other functions. Processor(s) 792 may be configured to receive data from the memory device(s) 793 and/or from the monitoring device 700, a tracking device, or another device. The speaker 796 may be used to provide audio alerts. Additionally, the speaker 796 may provide blood glucose levels in audio form for those with visual impairments, and the blood glucose levels may be converted from text or numbers to audio that is generated at the speaker 796. The light source 794 may comprise at least one of a light emitting diode (LED) light, an incandescent light, a fluorescent light, a halogen light, a laser, or other types of lights.

The monitoring device 700 also includes one or more processors 762. The processor(s) 762 may be configured to perform a variety of functions, and the processor(s) 762 may execute operations, computer readable code, or instructions stored at the memory device(s) 764 or at another location. Processor(s) 762 may cause presentation of images, information, symbols, or the like on the screen(s) 702. Processor(s) 762 may be configured to receive data from a tracking device or another device, cause the blood glucose levels or other health information to be presented on the screen(s) 702, and cause any other representations associated therewith to be changed in approximately real-time.

Processor(s) 768 of the external alert device(s) 766 may be configured to execute various instructions, computer readable code, or the like that are stored in the memory device(s) 770 of the external alert device(s) 766. Similarly, processor(s) 776 of the server(s) 774 may be configured to execute various instructions, computer readable code, or the like that are stored in the memory device(s) 778 of the server(s) 774. However, processor(s) 768 of the external alert device(s) 766 and processor(s) 776 of the server(s) 774 may be configured to execute various instructions stored in other memory devices.

The processor(s) 762, 768, 776, 792 may be any means configured to execute various programmed operations, computer readable code, instructions stored in a memory device (e.g., memory devices 764, 770, 778, 793) such as a device or circuitry operating in accordance with software or otherwise embodied in hardware or a combination of hardware and software (e.g. a processor operating under software control or the processor embodied as an application specific integrated circuit (ASIC) or field programmable gate array (FPGA) specifically configured to perform the operations described herein, or a combination thereof) thereby configuring the device or circuitry to perform the corresponding functions of the processor(s) 762, 768, 776, 792 as described herein.

The monitoring device 700 also includes memory device(s) 764. Similarly the memory device(s) 770 may be provided in the external alert device(s) 766 and the memory device(s) 778 may be provided in the server(s) 774. In an example embodiment, the memory devices 764, 770, 778, 793 may include one or more non-transitory storage or memory devices such as, for example, volatile and/or non-volatile memory that may be either fixed or removable. The memory devices 764, 770, 778, 793 may be configured to store instructions, computer program code, blood glucose level data, data related to insulin levels, other health data, location data, and additional data in a non-transitory computer readable medium for use, such as by the processor(s) 762, 768, 776, 792 for enabling the components of the system 701 to carry out various functions in accordance with example embodiments of the present invention. For example, the memory devices 764, 770, 778, 793 may be configured to buffer input data for processing by the processor(s) 762, 768, 776, 792. Additionally or alternatively, the memory devices 764, 770, 778, 793 may be configured to store instructions for execution by the processor(s) 762, 768, 776, 792. The memory devices 764, 770, 778, 793 may include computer program code that is configured to, when executed, cause processor(s) 762, 768, 776, 792 to perform various methods described herein. The memory devices 764, 770, 778, 793 may serve as non-transitory computer readable mediums having stored thereon software instructions that, when executed by one or more processors, cause methods described herein to be performed.

The communications interfaces 750, 772, 780, 795 may be configured to enable communication to other components of the system 701. For example, as information is obtained from tracking device(s) 746A-746N, the information may be received at any of the communications interfaces 750, 772, 780, 795 so that the information may be stored for later use or so that the information may be presented on the screen(s) 702, projected using one of projection device 790, or projection device(s) 760. In some embodiments, the information obtained from the tracking device(s) 746A-746N is sent first to the external alert device(s) 766 and/or the server(s) 774 for cloud storage before the information is sent to the monitoring device 700 or the projection device 790.

The communications interfaces 750, 772, 780, 795 may also include one or more communications modules configured to communicate with one another in any of a number of different manners including, for example, via a network. In this regard, the communications interfaces 750, 772, 780, 795 may include any of a number of different communication backbones or frameworks including, for example, Ethernet, global positioning system (GPS), cellular, Wi-Fi, or other suitable networks. The network may also support other data sources, including GPS. In this regard, numerous other peripheral devices may be included in the system 701. In some embodiments, some or all of the communications interfaces 750, 772, 780, 795 may be configured to communicate using short-range wireless technologies such as Bluetooth (e.g., Bluetooth Version 4.1 or another version), Wi-Fi, NearLink, near-field communication (NFC), low power wide area networks (LPWAN), ultra-wideband (UWB), wireless local area network (WLAN) in accordance with IEEE 802.11(b), IEEE 802.11(g), and/or IEEE 802.11(n) standards, and/or low-rate wireless personal access networks (LR-WPAN) pursuant to the IEEE 802.15.4 standard.

In the embodiment illustrated in FIG. 7, the system 701 comprises a number N different continuous tracking devices, with a first tracking device 746A, a second tracking device 746B, and additional tracking devices up to an Nth tracking device 746N. In some embodiments, systems may be provided with only one tracking device, but two or more tracking devices may be used in other embodiments.

While the system 701 is illustrated with various connections between components, these connections may be different in other embodiments. For example, there may be direct connections between the tracking device(s) 746A-746N and the server(s) 774. Additionally, the connections between the components may be wireless connections. For example, the monitoring device 700 may connect to other devices using a Bluetooth connection, a Wi-Fi connection, or another similar short range wireless technology. However, other connection approaches may be used for the monitoring device 700. Components such as the server(s) 774, the external alert device(s) 766, the projection device 790, and the tracking devices 746A-746N may be connected together in a wide variety of ways (e.g., Bluetooth, a cellular connection, another wireless connection, or the like). Connections may also be wired connections in some embodiments.

The system 701 may also comprise a controller device 775. The controller device 775 may be at least one of a disk controller, a graphics controller (GPU), a USB controller, a network controller, a memory controller, or the like, but the controller device 775 may be provided in various other forms. The controller device 775 may comprise a processor (e.g., a microcontroller), interface logic circuits, memory devices, buffers, and/or other components. The controller device 775 may comprise one or more output ports, and various devices may be connected to the output port(s). For example, as indicated by the dashed lines extending from the projection device 790 and the external alert device(s) 766 to the controller device 775, these devices 790, 766 may be connected to the controller device 775 at the output port(s). An output line (e.g., an output cable) may connect the signal output port of the monitoring device 700 to the controller device 775 at an output port. A first signal that causes the first adjustment in operation of the external device to be made may be configured to control flow of power and/or communications through the controller device to any devices attached to the controller device. In some embodiments, the controller device 775 may comprise a power supply independent of a power supply (e.g., power supply unit 705) for the monitoring device 700, and the controller device 775 is configured to turn power for the controller device 775 on or off to devices connected to the ports on the controller device 775 based on signals from ports of the monitoring device 700.

FIG. 8 is a schematic view illustrating various devices shown within different rooms within a home 800 and how monitoring device 808A may make changes to other devices based on data at the monitoring device 808A (e.g., through forward smart home integration) and how the monitoring device 808A may adjust its operation based on data from other devices (e.g., through reverse smart home integration). In some embodiments, monitoring devices may be integrated with other systems such as Google Home®, Apple Homekit™, or other similar systems. Forward smart home integration and reverse smart home integration may be accomplished using smart home integration protocols or other communication protocols or techniques.

An example home 800 is illustrated with a first room 802A and a second room 802B. Various devices may be provided in the two rooms 802A, 802B. For example, a first monitoring device 808A is provided in the first room 802A, and a second monitoring device 808B is provided in the second room 802B. These monitoring devices 808A, 808B may be similar to other monitoring devices 808A, 808B described herein. The monitoring devices 808A, 808B may be configured to connect with other devices in the home or outside of the home (see, e.g., device 899) to send and receive data with these other devices. The monitoring devices 808A, 808B may be configured to send data to other devices to adjust the operation of the other devices. Alternatively, the monitoring devices 808A, 808B may be configured to receive data from other devices, determine an adjustment in the operation of the monitoring devices 808A, 808B based on the received data, and cause the adjustment to be implemented. While monitoring devices 808A, 808B are illustrated in a home 800, the monitoring devices may be used in other locations (e.g., a workplace, a vehicle, a backyard, etc.).

The monitoring devices 808A, 808B may be configured to receive blood glucose level data for a tracked individual from a blood glucose monitor that the tracked individual is wearing. Based on the blood glucose level data, the monitoring devices 808A, 808B may each be configured to determine an adjustment in its operation based on the blood glucose level data. The monitoring devices 808A, 808B may also generate a signal to cause the adjustment to be made. Various adjustments may be implemented at the monitoring devices 808A, 808B based on the blood glucose level data that is received. For example, the image that is presented on a screen of the monitoring device may be adjusted, the font of text presented on the screen may be adjusted, the text color on the screen may be adjusted, the brightness of the material on the screen may be adjusted, the font size of text presented on the screen may be adjusted, a frequency at which material is flashed on the screen may be adjusted, the color of the background for the screen may be adjusted, the representation or slogan presented on the screen may be adjusted, or other adjustments may be implemented. Other data may be considered at the monitoring devices 808A, 808B.

Additionally, based on any blood glucose level data or other data received at the monitoring devices 808A, 808B, the monitoring devices 808A, 808B may each be configured to determine an adjustment in the operation of an external alert device, and the monitoring devices 808A, 808B may each be configured to generate a second signal that causes the second adjustment in operation of the external alert device to be made.

For example, the monitoring devices 808A, 808B may turn on lights in a particular room (e.g., a bedroom) if the blood glucose level falls too low. Monitoring devices 808A, 808B may also make more complex determinations about alerts to be made (e.g., through smart home integration)—for example, if blood glucose values are less than a particular level, a user is detected at home, and it is between 4 pm and 10 pm, then audio may be played on a speaker in the kitchen or the living room. In other examples, if the blood glucose values are less than a particular level, a determination may consider what rooms users are likely present in based on data received from other devices and/or historical data, the determination may consider the time of day, and then a particular type of alert may be generated in the appropriate room based on the time of day and other factors. For example, during the night, a bed shaker may be used to alert a user in some scenarios as this may be the most effective way to alert a user while the user is sleeping. During the day when a user is working, an alert may be generated at a phone or another display (e.g., on a television, a computer, a tablet, etc.) as this may be the most effective way to alert the user at that time. As another example, during the evening but before a user has gone to bed, an alert may be generated by making an alert in a particular room at the home of the user where the user is likely to be located (e.g., turning lights on or off, dimming lights, generating a sound indicative of an alert, generate an alert on a screen of a television, computer, tablet, smart phone, etc.).

Various external alert devices may be used in conjunction with the monitoring devices 808A, 808B, and some exemplary external alert devices are illustrated in the first room 802A and the second room 802B in FIG. 8. These external alert devices are external to the monitoring devices 808A, 808B. These external alert devices may include displays 804A, 804B (e.g., televisions), speakers 806A, 806B, lights 810A, 810B, fans 812A, 812B, heating and/or cooling devices 814A, 814B, vibration mechanisms 816A, 816B, projection devices 818A, 818B, and clocks 822A, 822B. Sensors 826A, 826B may also be positioned in the rooms 802A, 802B to detect certain conditions in the room. However, other external alert devices may also be used in conjunction with the monitoring devices 808A, 808B.

By causing adjustments in the operation of one or more of the external alert devices, the likelihood of successfully obtaining the attention of a user in the home 800 may be increased. These adjustments may be particularly beneficial where the user has a diminished ability to see, hear, feel, etc. When one of the user's senses are diminished in some way, then other sensory alerts designed to be detected by other senses of the user may be particularly beneficial to ensure that user detects the alert. For example, where a user is deaf, additional adjustments in the form of visual alerts generated on a display 804A, 804B may be beneficial. Where a user is blind, additional adjustments in the form of air blowing towards the user from a fan 812A or fan 812B, audio playing from speakers 806A, 806B, vibration from vibration mechanisms 816A, 816B, and/or other adjustments may increase the likelihood of the user noticing the issue. Also, when a user is sleeping, visual alerts may be less effective.

The monitoring devices 808A, 808B may cause several different types of adjustments in the operation of the external alert devices. The monitoring devices 808A, 808B may turn these external alert devices on or off (e.g., activating or deactivating the devices), adjust the magnitude at which the external alert devices are operated, or make other changes. The monitoring devices 808A, 808B may be configured to make adjustments to the external alert devices based on the blood glucose level data, and the adjustments may be changes in magnitude at the external device, a change in color at the external device, a change in frequency of an alert at the external device, or a change in a representation presented on the external device. However, other adjustments are also contemplated as discussed further herein.

Various types of input data may be considered in determining when, where, and how an alert is generated. For example, input data that is considered may comprise time data regarding a time of day, sensor data received from a sensor configured to detect a user in a room, usage data from the one or more external devices, or schedule data regarding a schedule of the user.

Input data that may be considered may also include duration data regarding a duration of time that an active alert has been unaddressed. When the duration data indicates that the active alert has been unaddressed for a longer duration, an alert may be generated with a greater magnitude. When the duration data indicates that the active alert has been unaddressed for a duration above a first time threshold, the alert may be generated at a different external device based on the duration data relative to when the duration data indicates that the active alert has been unaddressed for a duration below a first time threshold.

Depending on the input data received, the type of alert that is generated may be different. The alert type may be adjusted to adjust the external device where the alert is to be generated. For example, where a first dataset is used, an alert may be generated at a first external device, and when a second dataset is used that is different from the first dataset, an alert may be generated at a second external device. Where the input data indicates that a high urgency level is present, then an alert may be generated at a first external device, and when a lower urgency level is present, then an alert may be generated at a second external device.

Input data may be received for multiple tracked individuals in some embodiments, with first blood glucose level data being received for a first tracked individual and with second blood glucose level data being received for a second tracked individual. When an alert is generated based on the first blood glucose level data for the first tracked individual, the alert may be generated in a different manner relative to when an alert is generated based on the second blood glucose level data for the second tracked individual. For example, alerts for the first tracked individual may be generated at a first external device, and alerts for the second tracked individual may be generated at a second external device different from the first external device.

Displays 804A, 804B may be computer monitors, laptops, screens on a smartphone or tablet, a television, or some other sort of display. The displays 804A, 804B may be configured to display alerts or other information based on signals received from the monitoring devices 808A, 808B. For example, when a tracked individual has a blood glucose level that is far out of range, the monitoring devices 808A, 808B may cause an alert message to appear on the displays 804A, 804B.

In some embodiments, the displays 804A, 804B may act as pass through display devices, with these displays 804A, 804B being configured to present the same screen that is presented on the monitoring devices 808A, 808B. However, in some embodiments, the material presented on the displays 804A, 804B may differ slightly from the material presented on the monitoring devices 808A, 808B based on the size, resolution, or other properties of the displays 804A, 804B. In some embodiments, the displays 804A, 804B may include a window configured to present the same screen that is presented on the monitoring devices 808A, 808B, and the displays 804A, 804B may be configured to present other information as well.

The speakers 806A, 806B may be any sort of device configured to emit audio. The speakers 806A, 806B may be configured for use with a television, a computer, or any other device. The speakers 806A, 806B may be configured to generate audio alerts or other information based on signals received from the monitoring devices 808A, 808B.

While the lights 810A, 810B illustrated in FIG. 8 are illustrated on the ceilings 824A, 824B, the lights 810A, 810B may take various forms. For example, the lights 810A, 810B may be provided in the form of a lamp, lights on a Christmas tree, an LED string of lights, a light positioned behind a television or other furniture, a light on another device within the home, or the like. The lights 810A, 810B may be configured to add multiple effects such as sparkles, symbols bouncing back and forth, characters, symbols, text, slogans, or other effects.

In some embodiments, monitoring devices 808A, 808B may be configured to cause lights 810A, 810B to be turned on or turned off, to be dimmed or brightened, to change in color, to be flashed on or off briefly in some pattern indicative of an alert, or to be adjusted in some other manner. In some embodiments, the monitoring device may receive certain quantitative values for blood glucose level data and other data, and the monitoring devices may generate output signals to external devices with particular quantitative values rather than basic on or off signals. By doing so, external lights may be commanded to adjust to a particular brightness level or color based on the values in the output signals.

The fans 812A, 812B are illustrated as being portable fans resting on a floor in the rooms 802A, 802B. However, the fans 812A, 812B may be provided as ceiling fans or other types of fans in other embodiments. In some embodiments, monitoring devices 808A, 808B may be configured to cause the fans 812A, 812B to be turned on or turned off, to be rotated so that air is directed in a different direction, to be adjusted to increase or decrease the rotational speed of the fans 812A, 812B, or to be adjusted in some other manner. For example, a fan may be pointed towards a user, and, upon activation of an alert, the fan may be turned on to blow air on the user's face to help wake them or to otherwise grasp the user's attention. Activation of a fan due to alerts may be accomplished by using a power adapter, a USB adapter (e.g., a USB-C adapter), or an adapter may be implemented in the fan itself. For example, a wall plug adapter may be used that connects to a USB accessory port (e.g., a USB-C port) and that sits between the fan and a power outlet. The adapter may enable power to flow through it when the adapter receives an alert trigger, but the adapter may be configured so that it does not enable the flow of power through it when no alert trigger is received. Where a USB adapter or a USB-C adapter is used, the adapter may support a standard USB-powered device such as a lamp or a smaller fan, and the accessory port may provide power to the device when an alert is triggered instead of providing data that enables the external functionality.

The heating and/or cooling devices 814A, 814B are illustrated as resting on a floor in the rooms 802A, 802B. However, the heating and/or cooling devices 814A, 814B may be provided in other forms. The heating and/or cooling devices 814A, 814B may be configured to increase or decrease the temperature near a user. In some embodiments, the heating and/or cooling devices 814A, 814B may be used in conjunction with the fans 812A, 812B, thereby allowing the fans 812A, 812B to blow warmer air or colder air towards the user. In some embodiments, heating and/or cooling devices 814A, 814B may simply be a heating device without the ability to cool, heating and/or cooling devices 814A, 814B may simply be a cooling device without the ability to heat, or heating and/or cooling devices 814A, 814B may be used for heating and cooling. Monitoring devices 808A, 808B may be configured to cause devices 814A, 814B to be turned on or turned off, increased or decreased in magnitude, or adjusted in some other manner.

Vibration mechanisms 816A, 816B are illustrated on cabinets, the vibration mechanisms 816A, 816B may be positioned at other locations in the rooms 802A, 802B. Vibration mechanisms 816A, 816B may be any device configured to generate vibration. For example, the vibration mechanisms 816A, 816B may be provided in the form of a buzzer. Vibration mechanisms 816A, 816B may be configured to generate both vibration and sound in some embodiments to increase the likelihood of detection by users, particularly for users who are blind or those who are asleep. Vibration mechanisms 816A, 816B may be positioned at locations that may be the most likely to be detected by users when activated. For example, a vibration mechanism may be positioned on or underneath an armrest on a chair or a couch, at a nightstand, or at another location where the user typically is located at in the home 800. In some embodiments, monitoring devices 808A, 808B may be configured to cause vibration mechanisms 816A, 816B to be turned on or turned off, increased or decreased in magnitude, increased or decreased in frequency or magnitude of vibration, or adjusted in some other manner.

The projection device 818A may be configured to generate a first projection 820A towards the first ceiling 824A in the first room 802A, and the first projection 820A may form a projected image when the first projection 820A reaches the first ceiling 824A. Similarly, the projection device 818B may be configured to generate a second projection 820B towards the second ceiling 824B in the second room 802B, and the second projection 820B may form a projected image when the second projection 820B reaches the second ceiling 824B. While the projection devices 818A, 818B form projected images on the ceilings 824A, 824B, the projection devices 818A, 818B may be configured to generate projections 820A, 820B in different directions in other embodiments (e.g., so that projected images are formed on other walls in the home 800). In some embodiments, monitoring devices 808A, 808B may be configured to cause projection devices 818A, 818B to project a particular image, to project an alert, to flash an image or alert, to change in color, to change in direction, to change in font size or color for text in the projected image, to change an image that is projected, or to make some adjustment.

In some embodiments, the projection devices 818A, 818B may be configured to create waves of light that are projected towards the ceiling or a wall, with the waves of light resembling the Northern Lights. The waves that are generated may be adjusted in color based on the blood glucose level data, or the waves may be adjusted in some other manner (e.g., speed of waves, frequency of waves, or the like).

The clocks 822A, 822B may be digital clocks or clocks provided in other devices. In some embodiments, monitoring devices 808A, 808B may be configured to cause the clocks 822A, 822B to be adjusted so that they generate an alarm, turn on an audio feed (e.g., radio, music, or the like), flash an alert, present information, or vibrate. However, the monitoring devices 808A, 808B may cause different adjustments in the clocks 822A, 822B in other embodiments.

Various sensors 826A, 826B may also be provided in the home. These sensors 826A, 826B may include motion detection sensors, light sensors, electrical sensors configured to detect a voltage level, a power level, or a current level, or the sensors 826A, 826B may take some other form. Sensors 826A, 826B may determine whether a particular individual is located in a room and not sleeping. In some embodiments, the user of sensors 826A, 826B may allow for alerts to be generated in only the appropriate rooms, and this may allow for improved energy usage.

An external device 899 is also illustrated outside the home. This device 999 may be configured to send and receive signals with the monitoring device 808A and/or the monitoring device 808B so that adjustments may be made at the device 999 or at one or more of the monitoring devices 808A, 808B. The external device 899 may take a wide variety of different forms. For example, the device 899 may be a speaker outside of the home, a light, a device in a workplace of the user, a device in the vehicle of the user, or some other device. In some embodiments, the device 899 may be similar to another device described herein.

Where used, smart home integration (including forward smart home integration and reverse smart home integration) may be accomplished using existing hardware and without the need for any external device in some embodiments. Where this is the case, smart home integration may be implemented using one or more processors and/or memory devices within the monitoring device. In other embodiments, smart home integration may be accomplished using an external device. Where this is the case, the external device may have its own dedicated processor(s) and/or memory device(s) to assist with smart home integration. Smart home integration may be performed through the use of smart home integration protocols, but other protocols or techniques may be used to accomplish smart home integration.

In FIG. 8, example connections are shown in dashed lines. For example, the monitoring device 808A in the first room 802A is illustrated as being connected to the sensor 826A in the first room 802A, the sensor 826B in the second room, the light 810A in the first room 802A, the light 810B in the second room 802B, and to the monitoring device 808A in the second room 802B. However, these connections are merely exemplary, and the connections may be made with other devices as described herein.

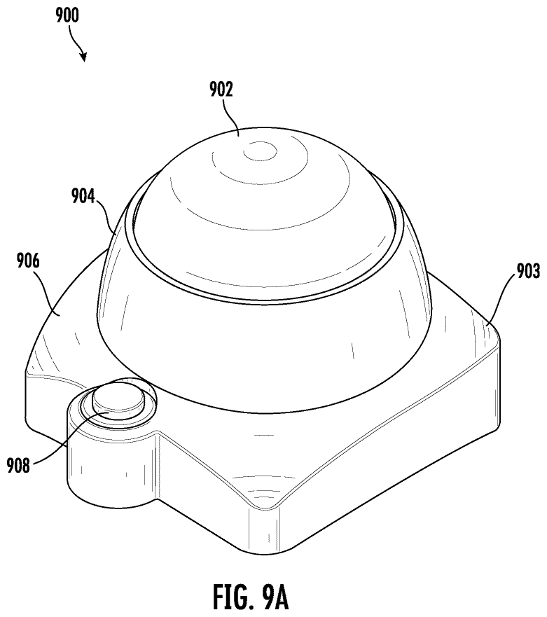

As described herein, projection devices may be used to project images towards a ceiling or towards another surface, and the images that are projected may be symbolic of a tracked individual's blood glucose level, health status, etc. FIGS. 9A-9B illustrate an example projection device, with FIG. 9A being a perspective view and with FIG. 9B being a rear view. In some embodiments, other projection devices described herein such as the projection device 790 of FIG. 7 or the projection devices 818A, 818B of FIG. 8 may be similar to the projection device 900 illustrated in FIGS. 9A-9B.

The projection device 900 may be configured to project an image towards a ceiling as described herein in reference to the projection devices 818A, 818B of FIG. 8. However, the projection device 900 may be configured to project images to other walls or surfaces. The projection device 900 may comprise a cover 902, a housing 903 having an upper portion 904 and a base portion 906, and a button 908.

The cover 902 has a spherical shape that resembles a dome, but the cover 902 may possess a different shape in other embodiments. The cover 902 may include a material that is at least partially transparent (e.g., glass), thereby allowing images to be projected through the cover 902. The housing 903 may comprise a material that is not transparent, thereby allowing light to only be emitted out of the projection device 900 at the cover 902. The cover 902 and the housing 903 may both be configured to retain other components (e.g., electrical components) within the projection device 900 and may protect these components from external conditions (e.g., humidity, dust, etc.). The upper portion 904 has a spherical curvature resembling the shape of the cover 902. However, the cover 902 and the housing 903 may possess different shapes and/or sizes in different embodiments.

The button 908 may be pressed by a user to adjust the operation of the projection device 900. For example, pressing the button 908 may cause the projection device 900 to be turned on or turned off or to adjust the projected image in some manner. In some embodiments, the manner in which the button 908 is pressed may impact the resulting output. For example, the projection device 900 may be turned on or turned off when the button 908 is pressed once, and the projected image projected from the projection device 900 may be adjusted when the button 908 is double tapped. In some embodiments, the resulting output from pressing the button may be dependent on the amount of time that the button 908 is pressed or based on other factors. Additionally, at surface 912, ports 914, 918, 920 are provided. The port 920 may be a USB-C port, and the port 918 may be an audio port, but other ports may also be used. However, a different number of ports may be used, ports may be provided with a different type, and/or ports may be provided at different locations on the projection device 900.

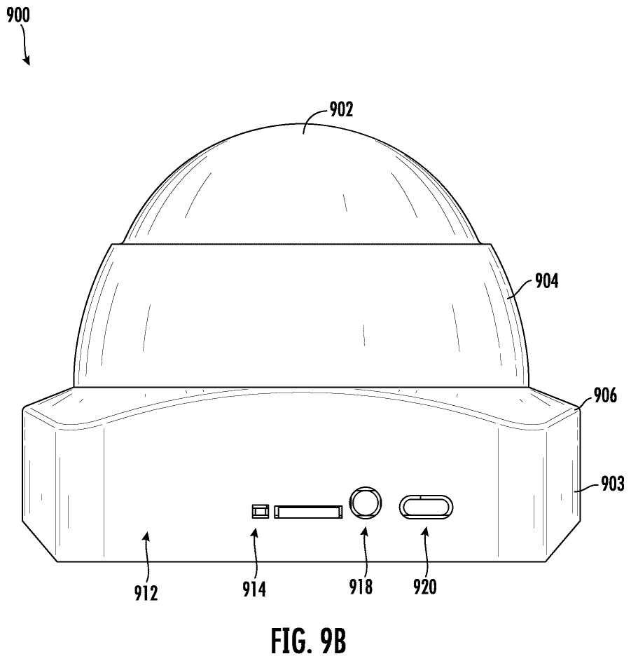

Connector devices are also contemplated that may assist users to safely and efficiently connect monitoring devices to other external devices. The connector devices may have a high compatibility with other external devices and may reduce the potential for user-error in making connections. FIG. 10 is a perspective view of an example connector device 1000 that may be used to connect a monitoring device and another external device. The connector device 1000 includes a first male connector 1002, a second male connector 1004, and a female connector 1006. In some embodiments, each of the connectors 1002, 1004, 1006 may be USB-C connectors.

The first male connector 1002 is configured to transfer power and data. The first male connector 1002 is attached to a first cable portion 1012, and this first cable portion 1012 extends between the first male connector 1002 and a junction 1008. In some embodiments, the first male connector 1002 may be configured to be plugged into monitoring devices described herein.

The second male connector 1004 is also configured to transfer power and data. The second male connector 1004 is attached to a second cable portion 1014, and this second cable portion 1014 extends between the second male connector 1004 and the junction 1008. The second male connector 1004 may be configured to be attached to an external device in some embodiments.

The female connector 1006 is only configured to transfer power and is not configured to transfer data. The female connector 1006 is attached to the third cable portion 1016, and this third cable portion 1016 extends between the female connector 1006 and the junction 1008.

The connector device 1000 may drastically reduce the risk of users plugging in external devices incorrectly. When the user only needs to use power without any external devices, then the user may plug in a standard USB-C cable into the female connector 1006. When the user is attempting to connect other external devices to a monitoring device, the first male connector 1002 may be connected to a USB-C port in the monitoring device and the second male connector 1004 may be connected to a USB-C port in the external device, thereby allowing data to be transferred between the monitoring device and the external device. The connector device 1000 may be configured to enable proper connections while reducing the risk of damage to electrical components.

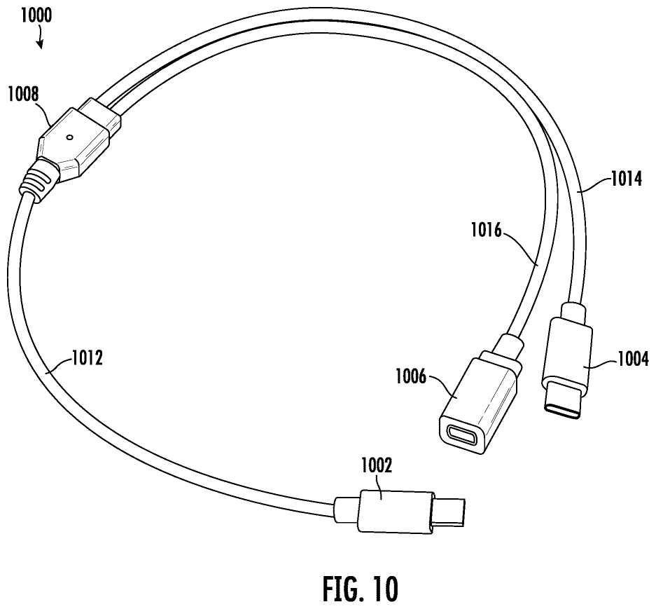

FIG. 11A includes schematic views of three different example external alert devices that may be vibrated to alert a user, and FIG. 11B is a schematic view illustrating the external alert devices of FIG. 11A attached to a bed frame 1112 of a bed 1110. The external alert devices 1100A-1100C of FIG. 11A may be positioned at various locations on a bed frame. These devices may vibrate to cause vibration of the bed frame, thereby increasing the likelihood that the user will detect an urgent alert when the user has been asleep.

An external alert device 1100A is illustrated, with this device having a cylindrical shape. The external alert device 1100A is provided in the form of a puck, but external alert devices may be provided in other forms and other shapes. The external alert device 1100A may be positioned beneath a leg of the bed frame 1112 so that the external alert device 1100A may vibrate the bed frame 1112. In FIG. 11B, the external alert device 1100A is positioned beneath only the first leg 1114A, and another object 1150 may be positioned underneath the second leg 1114B and/or other legs. However, external alert devices 1100A may be positioned under two or more of the legs of the bed. In some embodiments, one external alert device associated with a first tracked individual may be positioned under one side of the bed, and another external device associated with another tracked individual may be positioned under a second side of the bed—this may potentially allow one affected user/tracked individual to be woken up while another user/tracked individual in the bed remains asleep.

The external alert device 1100B comprises a body 1102 having a cylindrical shape similar to the external alert device 1100A, but the body 1102 comprises a recess 1104 therein. The external alert device 1100B may be received on any of the legs of the bed frame 1112. For example, in FIG. 11B, the external alert device 1100B is received on the second leg 1114B.

The external alert device 1100C comprises a fastener 1108 and a body 1106 having a rectangular prism shape. The fastener 1108 is provided in the form of a strap, but other fasteners may be used. For example, a hook and loop fastener, a magnet, adhesives, screws, bolts, mechanical snaps, etc. may be used. As illustrated in FIG. 11B, the external alert device 1100C is attached to the first leg 1114A of the bed frame 1112, with the fastener 1108 being wrapped around the first leg 1114A to hold the external alert device 1100C in position.

While the external alert devices 1100A-1100C are illustrated in certain positions in FIG. 11B, these devices may be positioned at other locations on the bed 1110 in other embodiments. For example, the devices may be positioned under a mattress but above the bed frame or the devices may be positioned at different locations on the bed frame. Additionally, the external alert devices 1100A-1100C may be provided at other locations to alert users. For example, these devices may be positioned on a chair, a couch, in a kitchen, or at other locations to alert a user. While the external alert devices 1100A-1100C are configured to vibrate to alert users, these devices may additionally or alternatively generate sound, lights, or some other output to get the user's attention. External alert devices 1100A-1100C may comprise electronic components similar to the external alert device(s) 766 of FIG. 7, and the external alert devices 1100A-1100C may function similarly to other external alert devices described herein in some embodiments.

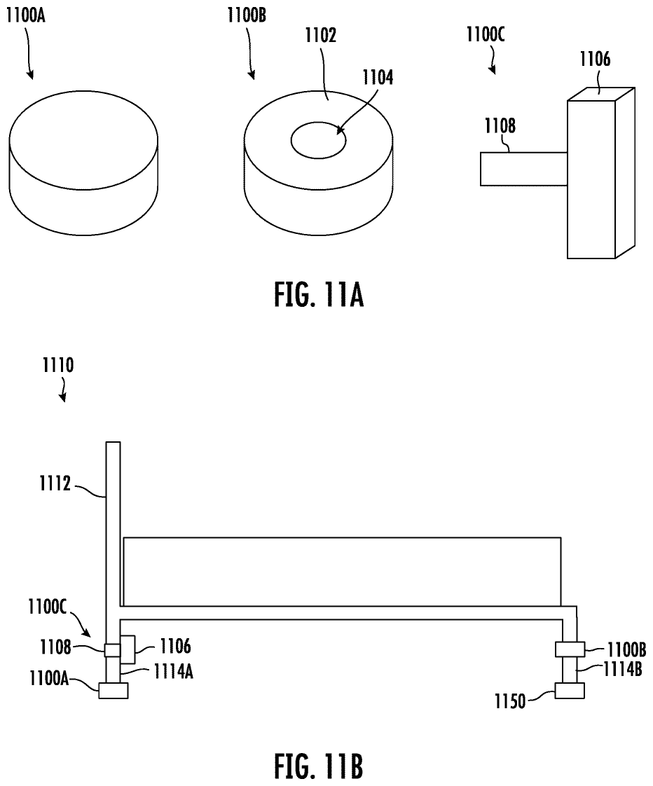

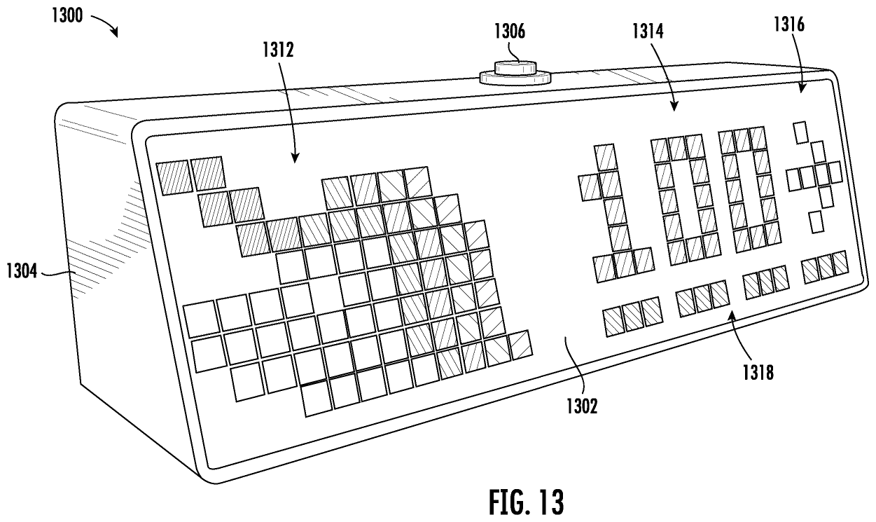

FIG. 12 is a schematic view of another external alert device 1200 that may be used to manage the operation of other devices. The external alert device 1200 is provided with the shape of a rectangular prism, but the device may have other shapes in other embodiments. The device 1200 may be configured to act like a controller. The device 1200 may receive pulses from monitoring devices described herein. The pulses may be communicated by a wired connection or via a wireless connection in some embodiments. Upon receiving the pulses, the device 1200 may determine what other external alert devices need to be adjusted and how the operation of these external alert devices need to be adjusted. For example, the device 1200 may receive a pulse, determine that it is necessary to adjust the operation of the external alert device 1400A of FIGS. 14A-14B, and then the device 1200 may cause the external alert device 1400A to vibrate to alert a user. In some embodiments, the device 1200 may receive a pulse, the device 1200 may determine which external alert device(s) should be powered on or off, and then the device 1200 may cause a signal to be sent to cause the external alert device(s) to be powered on or off. External alert device 1200 may comprise electronic components similar to the external alert device(s) 766 of FIG. 7, and the external alert devices 1200 may function similarly to other external alert devices described herein in many respects. In some embodiments, the external alert device 1200 and other external alert devices described herein may comprise their own power supply. The external alert device 1200 may comprise a single pin connector jack 1202 to help facilitate the connection of the device 1200 with other devices such as a monitoring device, but the external alert device 1200 may comprise ports such as a USB or a USB-C port.

In some embodiments, the external alert device 1200 may be configured to perform one or more of the various tasks described as being performed by a monitoring device herein. For example, rather than having certain determinations being made at a monitoring device, the alert device 1200 may instead be configured to perform these determinations in some embodiments. The alert device 1200 may be configured to control the generation of alerts at other devices. In some embodiments, the various monitoring devices may be configured to perform one or more of the various tasks described as being performed by an alert device herein (e.g., alert device 1200). For example, where the alert device 1200 is not used, a monitoring device may be configured to cause alerts to be generated at other devices when appropriate.

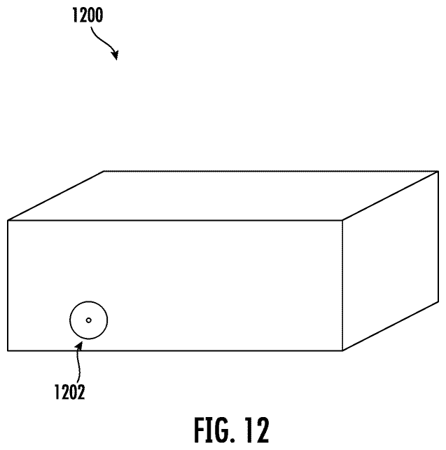

Another monitoring device 1300 is illustrated in FIG. 13 with information presented on the screen 1302 of the monitoring device 1300. The monitoring device 1300 may operate similarly to other monitoring devices described herein, and the monitoring device 1300 may include an outer shell 1304 and a button 1306 similar to the monitoring device 100 of FIGS. 1-6.

The monitoring device 1300 may display blood glucose levels for tracked individuals. The monitoring device 1300 illustrates a numerical indicator 1314, with this numerical indicator 1314 being the number one hundred. This numerical indicator 1314 is representative of the blood glucose level for the tracked individual, with the level being stated in milligrams per deciliter.

In the embodiment illustrated in FIG. 13, an indication 1312 is illustrated on the screen 1302. This indication 1312 is representative of the blood glucose level for the tracked individual, and the indication 1312 may be provided in the form of an emoji, icons, a word, a phrase. However, the indication 1312 may also be provided in other forms as well. In some embodiments, the indication 1312 may be a phrase that is presented. For example, the phrase may be “practice makes perfect” when the blood glucose level is at or near a perfect blood glucose level, the phrase may be “if you know you know” when the blood glucose level is not in the ideal blood glucose level range, and the phrase may be “mistakes were made” when the blood glucose level is in a very poor range.

In some embodiments, users may select their desired settings and set up for the monitoring device so that the information presented on the screen of the monitoring device is tailored as desired for the user preferences. For example, the user may make adjustments to select whether the monitoring device presents blood glucose levels for one tracked individual or for multiple tracked individuals. Settings may be adjusted to input user specific information such as the ideal blood glucose level(s) and other ranges of blood glucose level(s) that are less than ideal. For example, settings may be adjusted to have a ranges of Urgent Low, Low, In Range, High, and Urgent High. Where such ranges are used, blood glucose levels of less than about 60 may fall within the Urgent Low category, blood glucose levels between about 60 milligrams per deciliter and about 70 milligrams per deciliter may fall within the Low category, blood glucose levels between about 70 milligrams per deciliter and about 180 milligrams per deciliter may fall within the In Range category, blood glucose levels between about 180 milligrams per deciliter and about 250 milligrams per deciliter may fall in the High category, and blood glucose levels above about 250 milligrams per deciliter may fall in the Urgent High category (although other range values are contemplated). Alternatively, settings may be adjusted where the tracked individual has an A+ grade, an A grade, an A− grade, a B+ grade, a B grade, a B− grade, a C+ grade, and so forth. The A+ grade may correspond to blood glucose levels within five milligrams per deciliter of the ideal blood glucose level, the A grade may correspond to blood glucose levels outside of the A+ range but within ten milligrams per deciliter of the ideal blood glucose level, and so forth. However, other ranges may be used, and ranges may be adjusted by the user in some embodiments. For example, a simple grading range of A, B, C, D, F may be used. Additionally, different units may be used such as millimoles per liter or another unit rather than milligrams per deciliter. In some embodiments, rather than using an alphabetical grading system, other symbols, slogans, or indicators may be used. For example, as illustrated in FIG. 13, when the tracked individual has a perfect or near perfect blood glucose level, an indicator 1312 may appear with a unicorn to indicate a “unicorn” blood glucose level, and when the tracked individual has a very poor blood glucose level, other indicators 1312 may appear like a Grinch, Scrooge, a dumpster fire, etc.

In some embodiments, the indicator 1312 may be a user customized emoji, an icon, a slang term, a color, or some other indicator. For example, the indicator 1312 may be an emoji of a person clapping when the blood glucose level is perfect or near perfect, and the indicator 1312 may be a facepalm emoji when the blood glucose level is poor. As another example, the indicator 1312 may be a slang term or slogan such as “as good as it gets” when the blood glucose level is perfect or near perfect, and the indicator 1312 may be a slang term, slogan, or quote such as “not good enough” or “small daily improvements over time lead to stunning results.”

In some embodiments, the user may be able to create their own indicators (e.g., emojis, icons, slang terms, colors, etc.) or users may be able to select from a list of available indicators. However, in other embodiments, the indicators may be pre-set and may not be customizable by the user.

In some embodiments, the indicators 1312 may be color coded. For example, the indicators may be green when the blood glucose level is perfect or near perfect, indicators may be yellow when the blood glucose level is good but less than perfect, indicators may be orange when the blood glucose level needs improvement, and indicators may be red when the blood glucose level is very poor. The user may adjust the ranges of blood glucose levels corresponding to each color in some embodiments. Alternatively, in some embodiments, the color may gradually change based on changes in the blood glucose level rather than having ranges of blood glucose levels associated with different colors.

In the monitoring device 1300, the screen 1302 illustrates a number 1314, a trend arrow 1316, and a symbol 1318. The number 1314 may be the blood glucose level for a tracked individual, and the number 1314 may be presented differently depending on the blood glucose level and/or the rate of change for the blood glucose level. For example, in the monitoring device 1300, the number 1314 is presented in green text and in a relatively small font. The trend arrow 1316 serves as an indicator for the rate of change for the blood glucose level. The trend arrow 1316 is oriented horizontally to indicate that the blood glucose level remains relatively constant. Other symbols 1318 are also included to present further information. The symbols 1318 are indicative of the time remaining until the next blood glucose level data is received. For example, in the embodiment illustrated in FIG. 13, between 4 and 5 minutes have elapsed since the previous update, with each of the symbols 1318 representing an additional minute of time since the previous update.

Another example monitoring device 1400 is illustrated in the front, perspective view of FIG. 14. The monitoring device 1400 may operate similarly to other monitoring devices described herein, and the monitoring device 1400 may include a screen 1402, an outer shell 1404, and a button 1406 similar to the monitoring device 100 of FIGS. 1-6. The screen 1402 is presenting a number 1420, with the number 1420 indicating that the blood glucose level is about 189 milligrams per deciliter. Given the fact that the blood glucose level is relatively high and because the rate of change for the blood glucose level is increasing, the number 1420 and the trend arrow 1422 may be enlarged on the screen so that they are presented with greater emphasis, and the number 1420 may be presented in the color pink to further emphasize the blood glucose level.

In some embodiments, a trend arrow 1422 may be presented on the screen, with the trend arrow 1422 depending on the rate of change or the delta for the blood glucose level. The trend arrow 1422 may indicate how fast the blood glucose level is dropping or rising. The trend arrow 1422 may be beneficial to give a user a better understanding of the tracked individual's blood glucose levels. Where the trend arrow 1422 indicates a sharp increase in the blood glucose level and the blood glucose level is already relatively high, then this may be indicative of potential issues. Additionally, where the trend arrow 1422 indicates a decrease in the blood glucose level and the blood glucose level is relatively high, then this may be indicative that the blood glucose level is returning to a desired level.

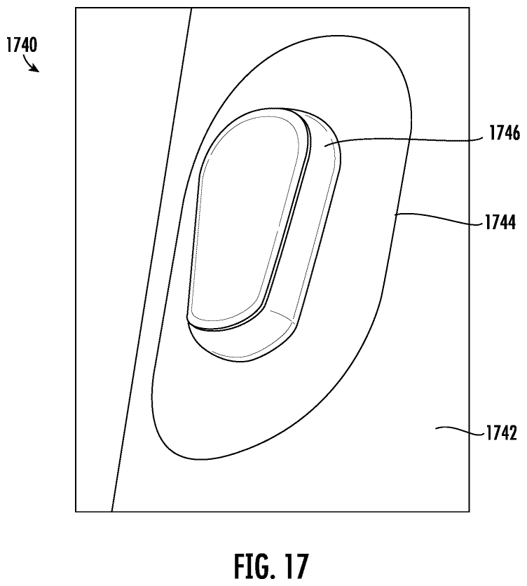

Turning ahead to FIG. 18, multiple graphs 1819A, 1819B are illustrated, with these graphs 1819A, 1819B illustrating the blood glucose level for tracked individuals over time. When the blood glucose levels are over the lower threshold 1823, the blood glucose levels may remain in a normal range. In some embodiments, the normal range may fall between an upper threshold and a lower threshold, but only a lower threshold 1823 is illustrated in the graphs 1819A, 1819B for the purposes of simplification.

In the graph 1819A, the plotline 1821A for the blood glucose level is initially in the normal range. However, the blood glucose level quickly approaches the lower threshold 1823, with the blood glucose changing at a constant rate (i.e., first derivative as a function of time). Given the relatively high rate of change (in terms of absolute value for the rate of change), an alert may be generated even before the blood glucose level falls below the lower threshold 1823. For example, an alert may be generated at a time corresponding to point 1825A. By considering the first derivative and/or the second derivative of the blood glucose level as a function of time in making alerts, alerts may be made earlier to help address issues swiftly. While the graph 1819A illustrates an alert being generated when the blood glucose level approaches a lower threshold for a normal range for the blood glucose level, an alert may similarly be generated where the blood glucose level approaches an upper threshold of a normal range for the blood glucose level.

Additionally, in some embodiments, other factors such as dehydration levels or other health related factors may be considered in determining whether it is appropriate to generate an alert. Readings from tracking devices may, at times, be inaccurate due to a variety of causes such as dehydration as an example. So, while a patient may otherwise have a low blood glucose level that falls out of the normal range, an alert may not be triggered because the dehydration levels indicate that the determined blood glucose level is not accurate. Monitoring devices and/or other devices may receive data from other sensors or other sources to identify and consider these issues when evaluating blood glucose levels.

In the graph 1819B of FIG. 18, the plotline 1821B for the blood glucose level is initially in the normal range, and the blood glucose level is approaching the lower threshold 1823. Eventually, the blood glucose level dips below the lower threshold 1823 proximate to point 1825B. However, in the illustrated example of the graph 1819B, the rate of change Δy/Δx (i.e., the first derivative as a function of time) of the blood glucose level at the point 1825B actually indicates that the blood glucose level is increasing. Given the rate of change Δy/Δx at the time corresponding to the point 1825B, a monitoring device may determine that no alert should be generated even though the blood glucose level is below the lower threshold 1823. Thus, in the example illustrated in the graph 1819B of FIG. 18, the consideration of the first derivative and the second derivative of the blood glucose level as a function of time may increase the accuracy of alerts so that alerts are only generated when necessary.

In some embodiments, the monitoring devices may consider the delta change value in determining whether to generate an alert, the type of alert, or the timing of the alert. For example, when the delta change value is relatively low to indicate that the change in blood glucose level is changing gradually, alerts may be provided less frequently or the monitoring devices may hold off on providing an alert. As another example, where the delta change value indicates that the blood glucose level is decreasing rapidly and that the blood glucose level is already very low, then alerts may be provided with high urgency, and alerts may be provided frequently.

Additionally, a delta threshold may be set. For example, the delta threshold may be set to around 5 milligrams per deciliter for a time thirty minutes, but other delta threshold values and corresponding times may be used, and the delta threshold and corresponding times may be customizable by the user in some embodiments. Where a delta threshold value is used, the blood glucose level may extend out of a specified range for a certain period of time. For example, a low threshold alert may be set to 70 milligrams per deciliter with a delta threshold of 5 milligrams per deciliter for 30 minutes. The alert may be ignored as long as the blood glucose level is higher than 65 milligrams per deciliter (the 70 milligrams per deciliter low threshold alert, minus the delta of 5 milligrams per deciliter). If the blood glucose drops below the allowed delta total of 65 milligrams per deciliter, the alert may immediately trigger. Or if the blood glucose level is still under 70 milligrams per deciliter after 30 minutes, the alert may be triggered. However, if the blood glucose level only extends into the blood glucose level range of 65 to 70 milligrams per deciliter for less than thirty minutes and then increases back above 70 milligrams per deciliter, then no alert may be triggered.

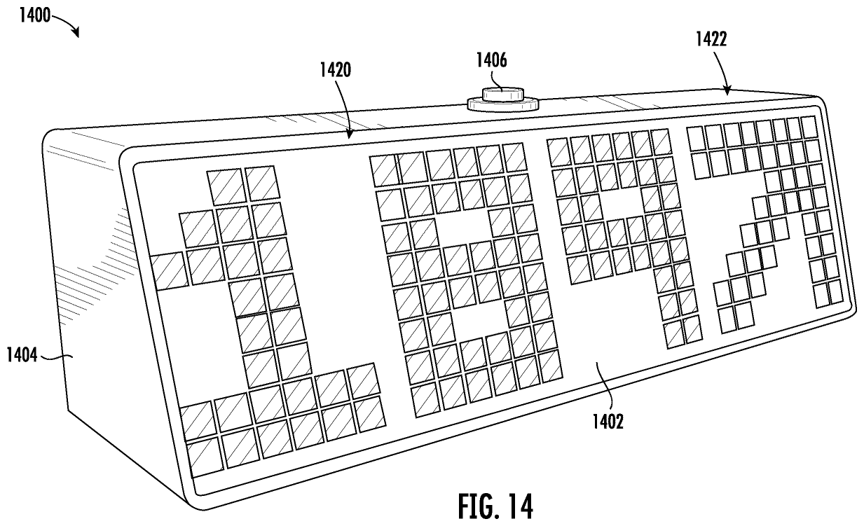

Another example monitoring device 1500 is illustrated in the front, perspective view of FIG. 15. The monitoring device 1500 may operate similarly to other monitoring devices described herein. In the example embodiment illustrated in FIG. 15, the monitoring device 1500 displays blood glucose levels on the screen 1502 for multiple people at same time. On the screen, a first number 1524 is presented for a first tracked individual, with the first number 1524 indicating that the blood glucose level for the first tracked individual is about 206 milligrams per deciliter. Additionally, a second number 1526 is presented for a second tracked individual, with the second number 1526 indicating that the blood glucose level for the second tracked individual is about 117 milligrams per deciliter. Additionally, a first trend arrow 1528 is illustrated on the screen 1500 to indicate that the blood glucose level for the first tracked individual is remaining approximately constant, and the second trend arrow 1530 is illustrated on the screen 1500 to indicate that the blood glucose level for the second tracked individual is increasing. The monitoring device 1500 may include a screen 1502, an outer shell 1504, and a button 1506 similar to the monitoring device 100 of FIGS. 1-6. The first number 1524 is presented in pink, and the second number 1526 is presented in green. These colors may be indicative of the tracked individuals so that the tracked individuals or other users (e.g., parents) may remember which number is associated with which tracked individual. Alternatively, the colors may be indicative of whether or not the blood glucose levels fall within the ideal range or farther away from the ideal range.

Where monitoring devices are configured to present information for multiple tracked individuals at the same time similar to monitoring device 1500, the monitoring device may be configured to cause alerts to be generated for each of the tracked individuals. Additionally, where monitoring devices are used to track multiple tracked individuals, then the alerts that are generated for the monitoring devices may be different depending on the tracked individual. For example, a first alert may be generated when the blood glucose levels are out of range for a first tracked individual but not the second tracked individual, a second alert may be generated when the blood glucose levels are out of range for a second tracked individual but not the first tracked individual, and a third alert may be generated when the blood glucose levels are out of range for both tracked individuals. In this example, each of the first, second, and third alerts may be different from each other to allow users to determine the tracked individual(s) that the alert applies to. The alerts may be different in terms of the sound that is generated by the monitoring device, but the alerts may also be generated by flashing the information on the screen or otherwise changing the presentation on the screen of the monitoring device.

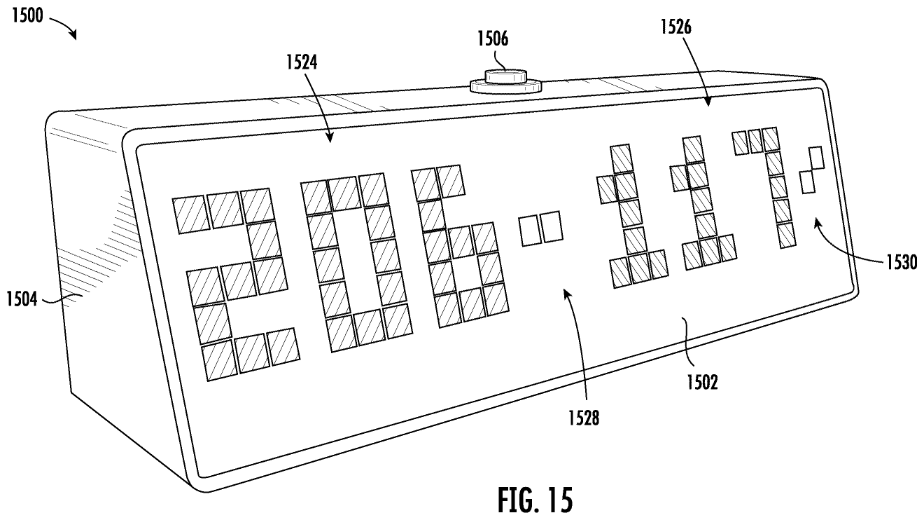

FIG. 16 is a front, perspective view illustrating an example monitoring device 1600 having a screen presenting different information. The monitoring device 1600 may operate similarly to other monitoring devices described herein. The monitoring device 1600 may include the screen 1602, an outer shell 1604, and a button 1606 similar to the monitoring device 160 of FIGS. 1-6.

On the screen 1602 of the monitoring device 1600, a number 1632 may be presented to indicate the blood glucose level for the tracked individual. The number 1632 indicates that the blood glucose level is about 119 milligrams per deciliter, and the number 1632 is presented in a green color. The screen 1602 also includes a trend arrow 1634 to indicate that the blood glucose level is relatively constant, and the indicator 1636 may show an indication of the change in blood glucose level over some duration of time. For example, the indicator 1636 indicates that the blood glucose level has changed by about 2 milligrams per deciliter over the previous minute. Additional symbols 1638 may also be included to present further information. For example, the symbols 1638 may represent the amount of time that has elapsed since the displayed reading, with each bar representing a minute that has elapsed. Thus, in the illustrated monitoring device 1600, the screen 1602 indicates that the 119 milligrams per deciliter reading was about 3 minutes ago. The individual pixels in the bars may represent subdivisions of a minute. For instance, each pixel may represent a twelve second increment and the five pixels in the bar total sixty seconds or one minute. The major continuous glucose monitors (CGMs) today update readings between every 1 minute and every 5 minutes. When a low blood glucose event happens, the amount of time from the previous reading may be critical information to know when to expect the next reading to see if you have had enough carbohydrates to correct the low glucose.

In some embodiments, the monitoring device 1600 or other monitoring devices described herein may be configured to present blood glucose levels over a certain period of time. For example, the monitoring devices may be configured to present a blood glucose level over a 1 minute interval, a 2 minute interval, a 5 minute interval, a 10 minute interval, a 15 minute interval, a 30 minute interval, a 60 minute interval, a three hour interval, a six hour interval, a twelve hour interval, a 24 hour interval, or some other interval. In some embodiments, the monitoring device 1600 or other monitoring devices described herein may be configured to present a daily time in range value for users. This daily time in range value may be the actual or approximate amount of time that the blood glucose levels for the user are within certain ranges. In some embodiments, these ranges may simply be a desired range and other ranges that fall outside of the desired range (e.g., above desired range, below desired range). The desired range may be preset in some embodiments without requiring user input, but the desired range may be adjusted in some embodiments to tailor the desired range based on the user's preferences. In some embodiments, more than three ranges may be monitored. For example, the ranges may include an extremely low blood glucose level range, a low blood glucose level range, a desirable blood glucose level range, a high blood glucose level range, and an extremely low blood glucose level range. In some embodiments, the ranges may be preset without requiring user input, but the desired range may be adjusted in some embodiments to enable a user to tailor the desired range based on the user's preferences.

In some embodiments, the user may receive a score based on the user's current blood glucose level, based on the user's most recent daily time in range values, and/or based on the user's daily time in range values over the course of the past several days, week(s), or month(s). The score may be presented in the form of the average or median daily time in range value in some embodiments, but the score may be provided in some other form such as an alphabetical score (A+, A, A−, B+, C, D, F, etc.), a numerical score (e.g., 1 to 100), etc. The score may be presented on a monitoring device 1600 or another monitoring device described herein. In some embodiments, the presentation on a screen of a monitoring device 1600 may be altered based on the user's score. For example, when the user's score is good, an emoji (e.g., a unicorn to represent a “unicorn” score), customizable term or slang term, or some other material may be presented to indicate that the score is good.

In some embodiments, material may be emphasized on the screen. Emphasis may be used to indicate when scores are good, but emphasis may also be used when blood glucose levels are poor and action is necessary to improve the blood glucose levels. For example, emphasis may be caused by flashing indicators or other information presented on the screen, by changing the color of the background of the screen, the text on the screen, the font size on the screen, the font of the text on the screen, etc. By providing emphasis, those with hearing impairments may be adequately alerted if any issues arise, and they may also have peace of mind that no issues are present even when alerts are not being presented.

In some embodiments, monitoring devices may be configured to adjust their appearance based on the time of day. For example, the monitoring devices may be connected to a clock to receive clock information, and the background color, the brightness of the screen, the color of certain text or indicators on the screen, or the amount of information presented on the screen may be adjusted based on the time of day. For example, during the evening during times when the user is likely sleeping, the monitoring devices may be changed to a night mode where the screen has a darker background, where text and indicators have reduced contrast, and/or where less information is presented on the screen. By contrast, during the day during times when the user is likely awake, the monitoring devices may be used in a normal mode where the screen has a brighter background, text and indicators that are brighter in color or that have more contrast, and/or more information presented on the screen. In some embodiments, when an alert is generated, the brightness, contrast, and/or presentation for material on the screen may be different even when the monitoring devices are operating in a night mode.

In some embodiments, the monitoring device may be presented in sleep mode at night, with no material presented on the screen. In some embodiments, the monitoring device may be configured so that it does not present any material on the screen during normal sleeping hours when in sleep mode and when the tracked individual's blood glucose levels are in a desired range, and the monitoring device may be configured so that it will present material on the screen during normal sleeping hours when in sleep mode and when the tracked individual's blood glucose levels are not in a desired range. The sleep mode may assist in reducing the energy consumption of the monitoring devices and may help to reduce distractions to the user as the user is trying to fall asleep. The sleep mode may also be used at other times of day, and the sleep mode is not limited to being used during normal sleeping hours in some embodiments.

In a similar vein, notifications may also be adjusted in operation based on the time of day. For example, alerts may be enabled at certain times of day, and alerts may be disabled at certain times of day. In some embodiments, the alert may be generated at a first external device at a first time of day, and the alert may be generated at a second external device different from the first external device at a second time of day different from the first time of day. The alert schedule may be set with times where alerts are enabled and disabled. The alert schedule may be preset by a manufacturer in some embodiments. The alert schedule may be set by default to generate alerts at all times of day when the monitoring devices are first sold. However, the alert schedule may be customizable by the user in some embodiments. Where the alert schedule is customizable by the user, the user may customize the alert schedule in hour increments, 30 minute increments, 15 minute increments, 10 minute increments, five minute increments, or one minute increments in some embodiments. However, the increments that the alert schedule may be customized in may be different in other embodiments. By allowing for modification of the alert schedule, the times may be easily adjusted when they may be less needed. For example, when the user is a child, a parent may adjust the times of operation so that the alerts are turned off when the child is typically at home during the day, and the alerts may be turned on at other times (e.g., when the child is at school, at a sporting event, when the child is asleep).

In some embodiments, monitoring devices may be used to display insulin pump data. For example, data may be presented to indicate whether the insulin pump is working appropriately, the battery level for the insulin pump, the amount of insulin on board, recent insulin doses, other data regarding the insulin pump.

Various features may be included to give the monitoring devices a selected theme. For example, the monitoring devices may be Halloween themed, Christmas themed, Easter themed, Thanksgiving themed, etc. However, the monitoring devices may also have other themes (e.g., superhero theme, sports theme, cartoon themed, etc.). The monitoring devices may include a projection device that is configured to project images towards a ceiling or towards another surface. The projection device may generate a single color, an image, a symbol, a number, etc., and the material that is generated may be dependent on the blood glucose level of the tracked individual. The projection device may generate themed images (e.g., northern lights images, Halloween theme, Christmas theme, Easter theme, Thanksgiving theme, etc.). A selected theme may also be implemented through the use of themed sounds (e.g., happy Christmas music or Christmas carols, scary music for Halloween). However, selected themes may be implemented through other ways such as the brightness of images presented, the color presented, the symbols presented, the typeface used for any text or numbers that are presented, and the like.

In some embodiments, the monitoring device may have connectivity to other lights such as lights on a Christmas tree, with the lights being configured to change in color. The color of the light that is emitted from the lights may be dependent on the blood glucose level of the tracked individual(s). For example, lights may be green when the blood glucose levels are good, yellow when blood glucose levels are in a moderate level, and red when the blood glucose levels are poor. In some embodiments, the monitoring device may cause the lights to flash repeatedly to alert the user(s) if the blood glucose level is poor or very poor.

The monitoring devices described herein may be used in a variety of different settings. For example, the monitoring devices may be utilized in a bedroom as an alarm clock or adjacent to an alarm clock, in an office, in a living room, in a workspace, a campsite, a recreational vehicle, or in a vehicle as a car dashboard device. However, the monitoring devices may also be used in other locations. The monitoring device may be configured to operate as an alarm clock.

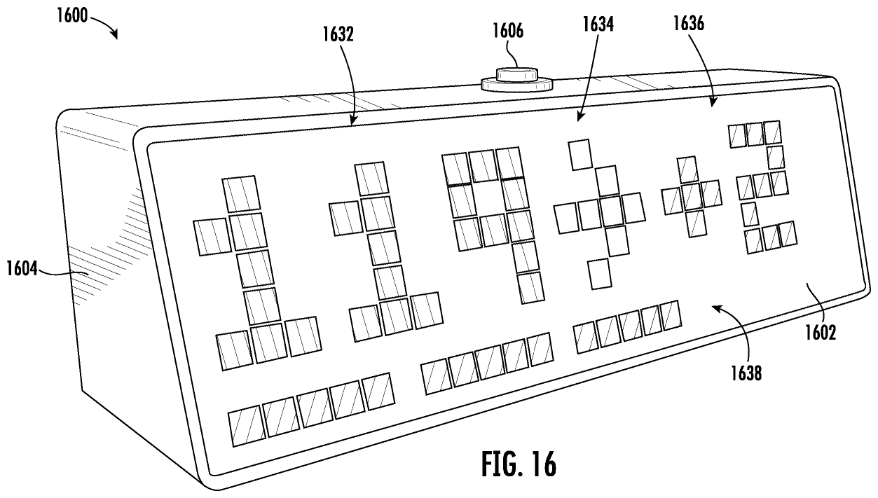

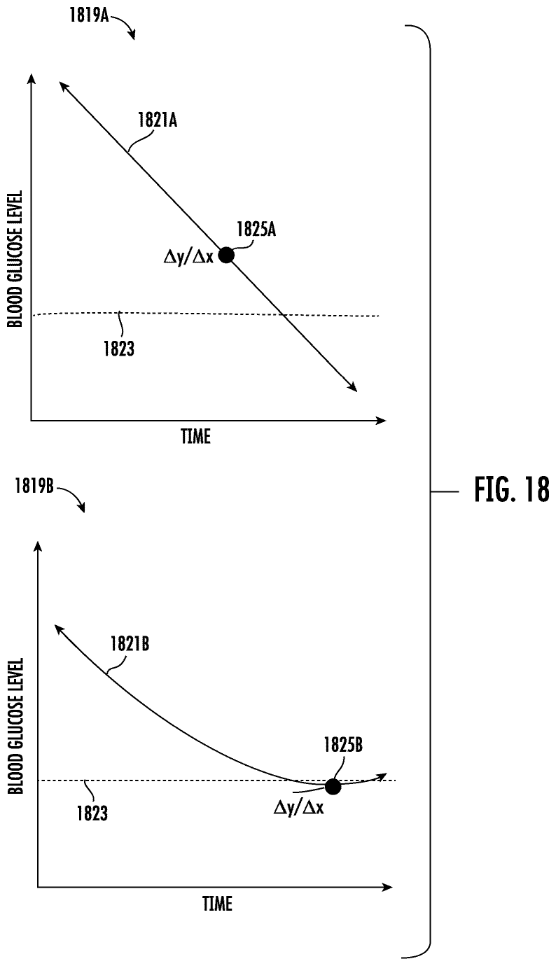

FIG. 17 illustrates a tracking device in the form of a continuous glucose monitor 1746 attached on an arm 1742 of a tracked individual 1740. The continuous glucose monitor 1746 may be attached using an attachment mechanism 1744, which may include an adhesive material. The continuous glucose monitor 1746 may obtain blood glucose level data for the tracked individual 1740, and the data from the continuous glucose monitor 1746 may be communicated to a smart phone for use on an application so that the data may then be communicated from the smart phone to the monitoring device for display. However, in some embodiments, a direct communication link may exist between the continuous glucose monitor 1746 and the monitoring devices described herein, or data from the continuous glucose monitor may be communicated to the monitoring device in other ways. FIG. 19 also illustrates an example of an insulin pump 1981 and an insulin pen 1983. The insulin pump 1981 may be configured to hold insulin and may include a cannula that goes under the skin to deliver insulin to the person wearing the insulin pump 1981. The insulin pump 1981 may be configured to send data related to the operation of the insulin pump 1981 to other devices. For example, the insulin pump 1981 may be sent to another device so that data associated with the insulin pump 1981 may be displayed. The insulin pen 1983 may be a smart insulin pen that is configured to track insulin doses, and the insulin pen 1983 may be configured to send this insulin dose data to other devices such as to a screen so that the insulin dose data or other data based on the insulin dose data may be displayed.

While various embodiments described herein are related to monitoring devices for monitoring blood glucose levels, it should be understood that the monitoring devices may additionally or alternatively be used to track other types of data or health data. For example, the monitoring devices may be used to track and present information related to a heart rate, a blood pressure, a distance traveled, a number of steps, a number of calories burned or consumed, the amount of carbohydrates consumed by the tracked individual during a meal or during a day, etc.

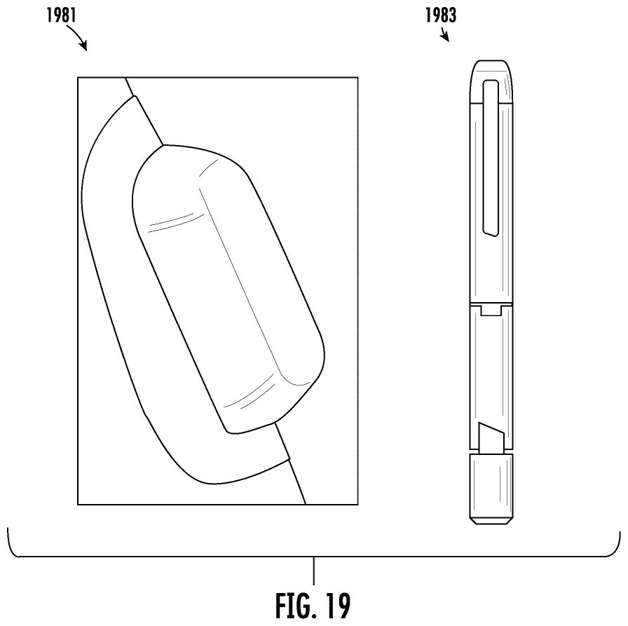

Methods are also contemplated for making changes to other devices in the home or elsewhere based on data at the monitoring device (e.g., through forward smart home integration) using monitoring devices. FIG. 20 is a flow chart illustrating an example method 2000 for adjusting an external alert device based on blood glucose level data.

At operation 2002, blood glucose level data may be received. The blood glucose level data may be received from a blood glucose monitor or from another device. The blood glucose monitor may be a continuous blood glucose monitor. In some embodiments, other data may also be received from the blood glucose monitor or other sources. Blood glucose data may also be received from other devices. For example, the blood glucose level data may be received from an application on a smart phone of a user.

At operation 2004, a first adjustment may be determined in the operation of a monitoring device based on the blood glucose level data. The first adjustment may also be determined based on other data such as other available health data, insulin levels, or the like.

The determination of the first adjustment and/or other adjustments may be made in various ways. For example, the determination may be made using pre-programmed algorithms or instructions, and these may be implemented based on expert recommendations or based on preferences of one or more users. In some embodiments, the determination may be made using artificial intelligence techniques or machine learning techniques. For example, models may be trained through supervised or unsupervised learning. Models may be developed based on anonymized training data, and the models may be configured to output determined adjustments that are appropriate. In some embodiments, any outputs from models may simply be provided as a recommendation to a user, and the user may then choose to authorize or reject the recommendation—the user may authorize the implementation of the recommendation in just that instance or in all similar situations in the future. However, in other embodiments, the recommendations developed from models may be automatically implemented.

At operation 2006, a first signal may be generated that causes the first adjustment in the operation of the monitoring device to be implemented. Various adjustments in the operation of the monitoring device may be made. For example, the image that is presented on a screen of the monitoring device may be adjusted, the font of text presented on the screen may be adjusted, the text color on the screen may be adjusted, the brightness of the material on the screen may be adjusted, the font size of text presented on the screen may be adjusted, a frequency at which material is flashed on the screen may be adjusted, the color of the background for the screen may be adjusted, the representation or slogan presented on the screen may be adjusted, or other adjustments may be implemented.

At operation 2008, a second adjustment may be determined in operation of an external alert device based on the blood glucose level data. While the first adjustment simply results in an adjustment in the operation of the monitoring device when implemented, the second adjustment, once implemented, may result in an adjustment in the operation of the external alert device, further increasing the likelihood of a user detecting an alert relative to other devices that only make adjustments at the monitoring device itself. The second adjustment may also be determined based on other data such as other available health data, insulin levels, or the like. The second adjustment may be determined using any of the techniques described herein for determinations of other adjustments.

At operation 2010, a second signal may be generated that causes the second adjustment in operation of the external alert device. As described herein, various adjustments may be made to the external alert device, and various external alert devices may be used. For example, various adjustments referenced in the discussion of FIG. 8 may be caused.

In some embodiments, the alert volume may be incremented so that the volume becomes incrementally louder over time. Alternatively, the first instance of an alert may simply trigger a vibration alert, and subsequent instances of an alert may trigger an audio alert and/or a vibration alert, with the alerts escalating in magnitude. Thus, if the user fails to address the alerts that are generated, the alerts may continue to get louder or may otherwise have a greater magnitude until the user addresses the issue or disables the alerts to indicate that the issue has been resolved. In some embodiments, the alerts may become louder or may have a greater magnitude after each minute that the alert is not snoozed, but the alerts may increase in volume or magnitude in different time intervals (e.g., once every 5 seconds, once every 15 seconds, once every 30 seconds, once every 2 minutes, once every 5 minutes, etc.). In some embodiments, an urgency level may increase the longer that an alert goes unaddressed.

In some embodiments, the user may receive an emergency communication if the user(s) do not address an alert within a specified time. This emergency communication may be a call or a text to a designated number, or the emergency communication may be an email sent to a designated email address. The user may be able to adjust the settings in some embodiments to enable or disable emergency communications, to enable or disable emergency communications at certain time windows, to change the time before any emergency communication is made, or to automate the emergency communication if an alert has not been snoozed within a certain time. In some embodiments, the monitoring device may be configured to make an emergency communication to another device if the alert has not been snoozed within a certain time, and this emergency communication may be provided in the form of a phone call, a text message, an email, a notification, etc.

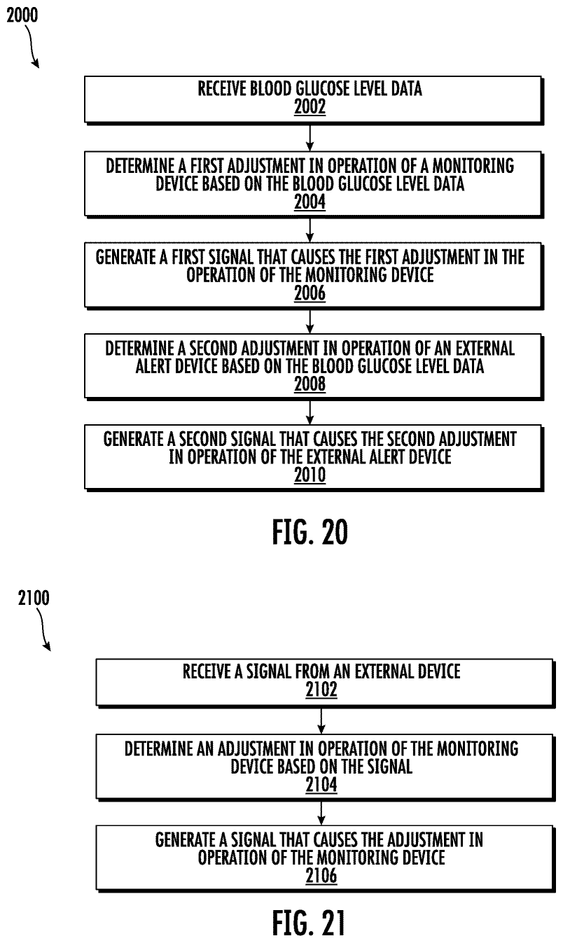

Methods are also contemplated for adjusting operation of a monitoring device based on data from other devices within the home or elsewhere (e.g., through reverse smart home integration), and FIG. 21 is a flow chart illustrating one such example method 2100. At operation 2102, a signal may be received from an external device. This signal may include data regarding the operation of the external device, regarding whether or not the external device is on, regarding occupancy of a room (e.g., by motion detection sensors or other devices), noise level, or other data.

At operation 2104, an adjustment in operation of the monitoring device may be determined based on the signal. Determinations may be made using any of the techniques described herein for determinations of other adjustments. Additionally, at operation 2106, a signal may be generated that causes the adjustment in operation of the monitoring device. Various adjustments may be made in the operation of the monitoring device based on the signals received from the external device. Example adjustments may include blocking an alarm from being generated at the monitoring device, causing an alarm to be generated at the monitoring device, changing a schedule when alarms are permitted at the monitoring device, changing a magnitude of an alert, changing a frequency of an alert at the monitoring device, changing a location where an alert is generated, indicating that there is a loss of data, or causing an adjustment at an external device that is external to the monitoring device. While various adjustments are contemplated in the generation of alerts at the monitoring device, adjustments may also impact the signals sent to other external alert devices by the monitoring device. For example, based on adjustments made at the monitoring device, the monitoring device may refrain from sending a signal to generate an alarm from being generated at another external alert device, cause an alarm to be generated at another external alert device, change a schedule when alarms are permitted at external alert devices, change a magnitude of an alert at another external alert device, change a frequency of an alert at another external alert device, or change a location where an alert is generated (e.g., generating an alert in a room that is occupied rather than one that is not occupied). Schedules may be changed to adjust quiet hours when alerts are permitted and not permitted-if devices in a home or in a particular room indicate that no user is present, then the monitoring device may intuitively adjust the quiet hours based on this data. These quiet hours may be provided in schedule data that is considered when determining the type of alert to be generated, the device where an alert is to be generated, etc. In some embodiments, if devices in a home or in a particular room indicate that no user is present, then the monitoring device may prevent an alert from being generated at the monitoring device or at other external alert devices. In some embodiments, if devices indicate that a user is present in a particular room, then the monitoring device may cause an external alert device in that room to be activated (or the monitoring device may generate an alert itself if the monitoring device is located in the room) while refraining from activating external alert devices in other rooms. This may improve energy usage by avoiding generation of alarms when users are not in the home or in a particular room.

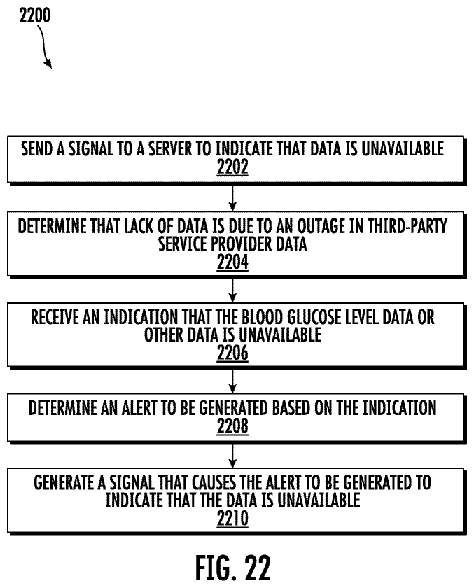

FIG. 22 is a flow chart illustrating an example method 2200 for generating alerts regarding a lack of availability for blood glucose level data or other data. At operation 2202, a signal may be sent to a server to indicate that data is unavailable at monitoring devices. This signal may be sent from a monitoring device or a processor in the monitoring device in some embodiments. In some embodiment, the signal may be sent directly or indirectly to one or more other monitoring devices.

At operation 2204, a determination may be made that the lack of data at monitoring devices is due to a widespread outage in third-party service provider data. This determination may be made at a server in some embodiments based on data received from a plurality of monitoring devices. For example, if a large number of monitoring devices are reporting an outage and the monitoring devices are each being used with a particular tracking device made by a particular manufacturer, then the server may determine that the manufacturer is currently experiencing an outage. Determinations may be made through algorithms or instructions, but the determinations may be made through the use of artificial intelligence and/or machine learning as described herein.

At operation 2206, an indication may be received that the blood glucose level data or other data is unavailable. The blood glucose level data or other data may be unavailable due to a third-party server being down, but this data may be unavailable for other reasons.

At operation 2208, a determination may be made on the alert to be generated based on the indication. When there is a large-scale outage in third-party service provider data, an alert may be generated at the monitoring device or at another device informing the user of the large-scale outage. In some embodiments, the alert may contain details regarding the outage and/or the status of repairs for the outage. Determinations may be made through algorithms or instructions, but the determinations may be made through the use of artificial intelligence and/or machine learning as described herein.

At operation 2210, a signal may be generated that causes the alert to be made to indicate that the blood glucose level data or other data is unavailable. The alert may also provide one or more potential causes for the issue and/or provide contact information to a third-party to get the issue resolved. These alerts may provide the user with peace of mind that his or her monitoring device is not in need of repair or replacement.

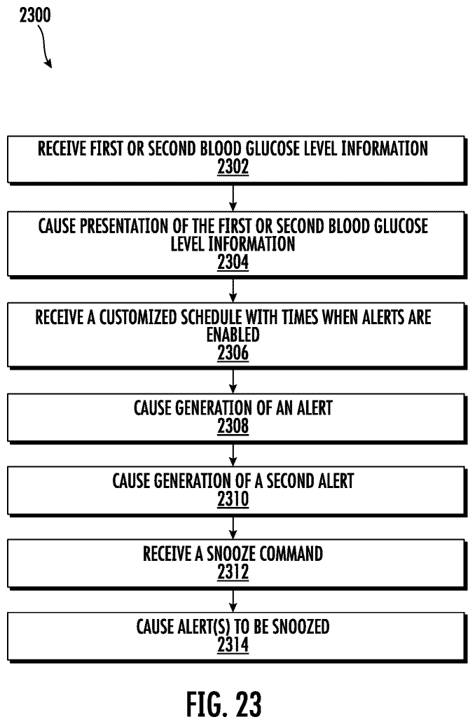

FIG. 23 illustrates an example flow chart with a method 2300 for presenting blood glucose level information for a tracked individual.

At operation 2302, first and/or second blood glucose level information may be received. The first blood glucose level information and the second blood glucose level information may each include at least one of a blood glucose level, a first derivative for the blood glucose level as a function of time, or a second derivative for the blood glucose level as a function of time. The first and second blood glucose level information may be received from a respective tracking device, with the tracking device being a continuous blood glucose monitor, an insulin pump, a smart insulin pen, or a similar device. Where first and second blood glucose level information are received, this information may be for two different tracked individuals (e.g., two children of a parent).

In some embodiments, the first and/or second blood glucose level information may include data having specific blood glucose level information. However, in some embodiments, the first and/or second blood glucose level information may also include indication that the blood glucose level either falls inside or outside of an acceptable range, with these indications not including any specific blood glucose level. These indications may be received at a more regular frequency than the frequency at which data having specific blood glucose level information is received, allowing alerts to be generated more quickly if a tracked individual is outside of an acceptable range.

At operation 2304, presentation of the first and/or second blood glucose level information may be caused. For example, presentation may be caused on a screen of a monitoring device or at another location.

At operation 2306, a customized schedule may be received with times when alerts are enabled. By doing so, alerts may only be generated during time periods when the user's customized schedule allows alerts, and alerts may be prevented during other time periods. However, in some embodiments, even when the user has disabled alerts during a particular time, alerts may still be generated if the tracked individual triggers the alert or if the blood glucose level information is extremely poor.

At operation 2308, generation of an alert may be caused. This may be caused based on the first and/or the second blood glucose level information, and the alerts may include audio alerts, visual alerts, vibratory alerts, or other similar alerts.

At operation 2310, generation of a second alert may be caused. The alerts may be randomized to prevent brain habituation—for example, the second alert that is generated may be distinct from the first alert (e.g., in its sound, color, frequency, etc.). In some embodiments, if a user has not snoozed an alert after a certain duration of time, a second alert having a greater magnitude may be generated. The magnitude may be greater in terms of volume level, brightness, flashing frequency, prominence of a visual alert display, or in other ways. In some embodiments, if a user has not snoozed an alert after a certain duration of time, a second alert may be caused that sends a notification to another device, with the notification including a phone call, a text message, an email, and/or a notification alert.

At operation 2312, a snooze command may be received. In some embodiments, this snooze command may be received at a separate device from the monitoring devices described herein, such as from a remote computer or phone. However, in some embodiments, snooze commands may simply be received at a monitoring device.

At operation 2314, snoozing of one or more alerts may be caused. In some embodiments, snoozing may occur only at one monitoring device. However, in other embodiments, snoozing may occur at multiple monitoring devices or at other devices where alerts may be triggered (e.g., a computer, a phone, etc.).

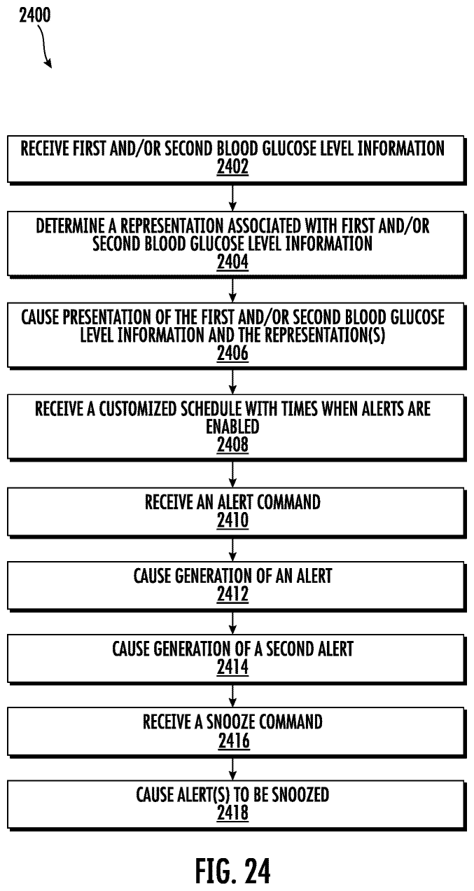

FIG. 24 illustrates an example flow chart with a method 2400 for presenting blood glucose level information for a tracked individual. At operation 2402, first blood glucose level information and/or second blood glucose level information may be received. The first blood glucose level information and the second blood glucose level information may each include at least one of a blood glucose level, a first derivative for the blood glucose level as a function of time, or a second derivative for the blood glucose level as a function of time. The first and second blood glucose level information may be received from a respective tracking device, with the tracking device being a continuous blood glucose monitor, an insulin pump, a smart insulin pen, or a similar device. Where first and second blood glucose level information are received, this information may be for two different tracked individuals (e.g., two children of a parent). In addition to the blood glucose level information, data from an insulin pump or a smart insulin pen may be received and displayed on devices.

In some embodiments, the first and/or second blood glucose level information may include data having specific blood glucose level information. However, in some embodiments, the first and/or second blood glucose level information may also include indication that the blood glucose level either falls inside or outside of an acceptable range, with these indications not including any specific blood glucose level. These indications may be received at a more regular frequency than the frequency at which data having specific blood glucose level information is received, allowing alerts to be generated more quickly if a tracked individual is outside of an acceptable range. The first and/or the second blood glucose level information may first be sent to a user device and/or a server before it is received at the monitoring device. The user device may be at least one of a computer, a cell phone, a smart phone, receivers, or a tablet.

At operation 2404, a representation associated with the first blood glucose level information and/or the second blood glucose level information may be determined. This determination may be made based on the first blood glucose level information and/or the second blood glucose level information received at operation 2402. The representation may include at least one of an emoji, a slang term, a color, a quote, or something similar. In some embodiments, the representation(s) may be customizable by a tracked individual, a user, or by another person so that customized representations may be presented for different blood glucose level ranges.

At operation 2406, presentation of the first and/or the second blood glucose level information and the associated representation(s) may be caused. Presentation of this material may be caused on the screen of a monitoring device, but presentation of this material may occur at another location in some embodiments.

At operation 2408, a customized schedule may be received with times when alerts are enabled. By doing so, alerts may only be generated during time periods when the user's customized schedule allows alerts, and alerts may be prevented during other time periods. However, in some embodiments, even when the user has disabled alerts during a particular time, alerts may still be generated if the tracked individual triggers the alert or if the blood glucose level information is extremely poor.

At operation 2410, an alert command may be received. This may be received by a tracked individual on a tracking device that the tracked individual is wearing, by the tracked individual or another user on a monitoring device, or by a tracked individual or another user on another device. The alert command may be generated upon an alert button being pressed by a user in some embodiments, but the alert command may be generated upon a voice command or some other method of actuation.

At operation 2412, generation of an alert may be caused, and the alert may be based on the first and/or the second blood glucose level information. The alert may comprise at least one of an audio alert, a visual alert, or a vibratory alert. The alert may be caused at a particular monitoring device, at another monitoring device (and not one where an alert command may have been triggered), or at another device.

At operation 2414, generation of a second alert may be caused. The alerts may be randomized to prevent brain habituation—for example, the second alert that is generated may be distinct from the first alert (e.g., in its sound, color, frequency, etc.). In some embodiments, if a user has not snoozed an alert after a certain duration of time, a second alert having a greater magnitude may be generated. The magnitude may be greater in terms of volume level, brightness, flashing frequency, prominence of a visual alert display, or in other ways. In some embodiments, if a user has not snoozed an alert after a certain duration of time, a second alert may be caused that sends a notification to another device, with the notification including a phone call, a text message, an email, and/or a notification alert.

At operation 2416, a snooze command may be received. In some embodiments, this snooze command may be received at a separate device from the monitoring devices described herein, such as from a remote computer or phone. However, in some embodiments, snooze commands may simply be received at a monitoring device.

At operation 2418, one or more alerts may be snoozed. In some embodiments, snoozing may occur only at one monitoring device. However, in other embodiments, snoozing may occur at multiple monitoring devices or at other devices where alerts may be triggered (e.g., a computer, a phone, etc.).

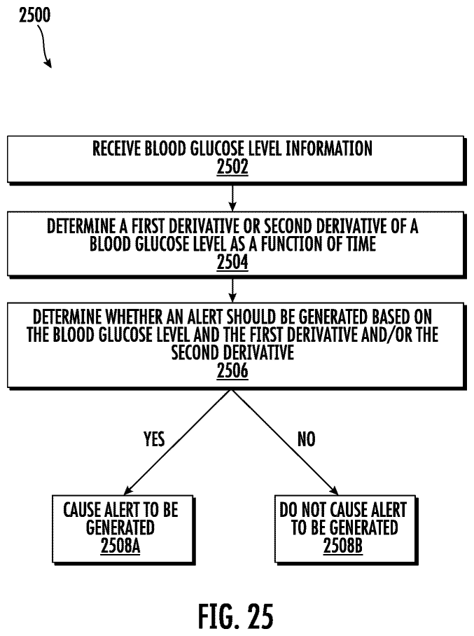

FIG. 25 illustrates an example flow chart with a method for presenting blood glucose level information for a tracked individual. At operation 2502, blood glucose level information may be received. The blood glucose level information may include a blood glucose level. The blood glucose level information may be received from a continuous blood glucose monitor, but other information or data may also be received at operation 2502, with this information or data being received from an insulin pump or a smart insulin pen.

At operation 2504, a determination of the first derivative and/or the second derivative of the blood glucose level as a function of time may be determined. Additionally, at operation 2506, a determination may be made as to whether an alert should be generated based on the blood glucose level and the first derivative and/or the second derivative. For example, a determination may be made that the alert should be generated when the blood glucose level is in a normal range but the first derivative and/or the second derivative for the blood glucose level indicate that the blood glucose level is about to exit the normal range. As another example, a determination may be made that the alert should not be generated where the blood glucose level falls outside of a normal range but a first derivative for the blood glucose level indicates that the blood glucose level is about to enter into the normal range. If it is determined that an alert should be generated at operation 2506, then the method 2500 may proceed to operation 2508A. If it is determined that no alert should be generated at operation 2506, then the method may proceed to operation 2508B. At operation 2508A, generation of an alert may be caused as described herein. At operation 2508B, generation of an alert may not be caused.

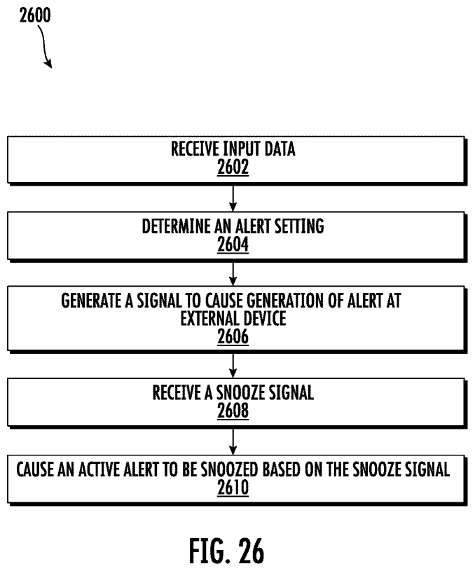

Another example method for generation of alerts at one or more external devices is illustrated in FIG. 26. At operation 2602, input data is received. The input data comprises blood glucose level data, and this blood glucose level data may be received from a tracking device such as a blood glucose monitor. In some embodiments, the input data may comprise time data regarding a time of day. Additionally or alternatively, the input data may comprise sensor data received from a sensor configured to detect a user in a room, usage data from the one or more external devices, duration data regarding a duration of time that an active alert has been unaddressed, or schedule data regarding a schedule of the user.

At operation 2604, an alert setting is determined. The alert setting may be for an alert to be generated at an external device, and the alert setting may be determined based on the based on the input data. The alert setting may include at least one of an identification of the external device where the alert is to be generated, a magnitude of the alert, or an alert type, and the alert type may be at least one of an audible alert, a haptic alert, or a visual alert. The external device may comprise at least one of a monitoring device for presenting blood glucose level information for a tracked individual, a light, a fan, a vibration mechanism, a television, a projection device configured to project an image, a clock, a heating device, a cooling device, an audio device, a device capable of sending a message, or a device capable of making an audio or a video call.

Where the alert is a visual alert, the visual alert may comprise a notification on a screen or a projection from a projector, an image on a screen or a projection from a projector, an emoji, flashing lights, activation or deactivation of a light, dimming or brightening of a light, or a change in color on a device. However, other types of visual alerts may be generated in some embodiments.

Where the alert is a haptic alert, the haptic alert may cause vibration of one or more external devices or activation of a fan to blow air. For example, the fan may be activated to blow air towards a user to alert the user of an issue.

At operation 2606, a signal is generated to cause generation of an alert at an external device, and the alert has an alert type.

At operation 2608, a snooze signal is received.

At operation 2610, an active alert is snoozed based on the snooze signal. In some embodiments, the active alert may be snoozed at only one external device. However, in other embodiments, active alerts may be snoozed at multiple devices. Additionally, in some embodiments, active alerts for different tracked individuals may be snoozed at one time.

In some embodiments, the methods described herein may be implemented using one or more processors. In some embodiments, computer readable code may be stored on one or more memory devices, and this computer readable code may be configured to cause the processor(s) to perform one or more of the methods described herein. The methods 2000, 2100, 2200, 2300, 2400, 2500, 2600 are each exemplary, and these methods may be modified in other embodiments. For example, the operations of these methods may be performed in different orders, and some of the operations may be performed simultaneously in some embodiments. The methods may be combined together in some embodiments. Furthermore, certain operations within the methods may be omitted and additional operations may be added.

### CONCLUSION

Many modifications and other embodiments set forth herein will come to mind to one skilled in the art to which these embodiments pertain having the benefit of the teachings presented in the foregoing descriptions and the associated drawings. Therefore, it is to be understood that the embodiments are not to be limited to the specific embodiments disclosed and that modifications and other embodiments are intended to be included within the scope of the invention. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the invention. In this regard, for example, different combinations of elements and/or functions than those explicitly described above are also contemplated within the scope of the invention. Although specific terms are employed herein, they are used in a generic and descriptive sense only and not for purposes of limitation.

What is claimed is:

1. An alert device for management of alerts generated at one or more external devices, the alert device comprising:
at least one processor; and
at least one memory device comprising computer readable code that, when executed by the at least one processor, is configured to cause the at least one processor to:
receive input data, wherein the input data comprises blood glucose level data for one or more tracked individuals;
determine an alert setting for an alert to be generated at an external device of the one or more external devices based on the input data, wherein the alert setting includes at least one of:
an identification of the external device where the alert is to be generated;
a magnitude of the alert; or
an alert type, with the alert type being at least one of an audible alert, a haptic alert, or a visual alert; and
generate a signal that causes the alert to be generated at the external device, with the alert having the alert type.

2. The alert device of claim 1, wherein the input data comprises at least one of:
time data regarding a time of day;
sensor data received from a sensor configured to detect a user in a room;
usage data from the one or more external devices;
duration data regarding a duration of time that an active alert has been unaddressed; or
schedule data regarding a schedule of the user.

3. The alert device of claim 2, wherein the schedule data comprises data regarding quiet hours for the user when certain alerts are disabled.

4. The alert device of claim 2, wherein, when the duration data indicates that the active alert has been unaddressed for a longer duration, the at least one processor causes the alert to be generated at the external device with a greater magnitude.

5. The alert device of claim 2, wherein, when the duration data indicates that the active alert has been unaddressed for a duration above a first time threshold, the at least one processor causes the alert to be generated at a different external device based on the duration data relative to when the duration data indicates that the active alert has been unaddressed for a duration below a first time threshold.

6. The alert device of claim 1, wherein the alert setting includes the alert type, the alert type is a first alert type when the input data comprises a first dataset, and the alert type is a second alert type when the input data comprises a second dataset different from the first dataset.

7. The alert device of claim 6, wherein the alert setting includes the identification of the external device where the alert is to be generated, the external device is a first external device when the input data comprises the first dataset, and the external device is a second external device different from the first external device when the input data comprises the second dataset.

8. The alert device of claim 1, wherein the external device is a first external device when the input data comprises a first dataset, and the external device is a second external device different from the first external device when the input data comprises a second dataset.

9. The alert device of claim 8, wherein the external device comprises at least one of a monitoring device for presenting blood glucose level information for a tracked individual, a light, a fan, a vibration mechanism, a television, a projection device configured to project an image, a clock, a heating device, a cooling device, an audio device, a device capable of sending a message, or a device capable of making an audio or a video call.

10. The alert device of claim 1, wherein the blood glucose level data is received from a blood glucose monitor.

11. The alert device of claim 1, wherein the input data comprises first blood glucose level data for a first tracked individual and second blood glucose level data for a second tracked individual, the at least one processor causes the alert to be generated in a first manner when an alert is associated with the first blood glucose level data for the first tracked individual, and the at least one processor causes the alert to be generated in a second manner different from the first manner when the alert is associated with the second blood glucose level data for the second tracked individual.

12. The alert device of claim 11, wherein the at least one processor causes the alert to be generated at a first external device when an alert is associated with the first blood glucose level data for the first tracked individual, and the at least one processor causes the alert to be generated at a second external device when the alert is associated with the second blood glucose level data for the second tracked individual.

13. The alert device of claim 1, wherein the identification of the external device where the alert is to be generated is determined by determining an urgency level based on the input data.

14. The alert device of claim 13, wherein the at least one processor causes the alert to be generated at a first external device when a first urgency level is present, and the at least one processor causes the alert to be generated at a second external device when a second urgency level greater than the first urgency level is present.

15. The alert device of claim 1, wherein the at least one processor causes the alert to be generated at a first external device at a first time of day, and the at least one processor causes the alert to be generated at a second external device different from the first external device at a second time of day different from the first time of day.

16. The alert device of claim 1, wherein, when executed by the at least one processor, the computer readable code is configured to cause the at least one processor to:
receive a snooze signal; and
cause an active alert at the one or more external devices to be snoozed based on the snooze signal.

17. The alert device of claim 1, wherein the visual alert comprises a notification on a screen or a projection from a projector, an image on a screen or a projection from a projector, an emoji, flashing lights, activation or deactivation of a light, dimming or brightening of a light, or a change in color on a device.

18. The alert device of claim 1, wherein the haptic alert results in vibration of one or more of the external devices or activation of a fan to blow air.

19. A system for generation of alerts at one or more external devices, the system comprising:
the one or more external devices;
one or more tracking devices configured to generate blood glucose level data for one or more tracked individuals;
an alert device for management of alerts generated at the one or more external devices, the alert device comprising:
at least one processor configured to:
receive input data, wherein the input data comprises blood glucose level data received from the one or more tracking devices;
determine an alert setting for an alert to be generated at an external device of the one or more external devices based on the input data, wherein the alert setting includes at least one of:
an identification of the external device where the alert is to be generated;
a magnitude of the alert; or
an alert type, with the alert type being at least one of an audible alert, a haptic alert, or a visual alert; and
generate a signal that causes the alert to be generated at the external device, with the alert having the alert type.

20. A method for generation of alerts at one or more external devices, the method comprising:
receiving input data, wherein the input data comprises blood glucose level data received from a tracking device;
determining an alert setting for an alert to be generated at an external device of the one or more external devices based on the input data, wherein the alert setting includes at least one of:
an identification of the external device where the alert is to be generated;
a magnitude of the alert; or
an alert type, with the alert type being at least one of an audible alert, a haptic alert, or a visual alert; and
generate a signal that causes the alert to be generated at the external device, with the alert having the alert type.
